(19) United States
(12) Patent Application Publication
(10) Pub. No.: US 2011/0112434 A1
(43) Pub. Date: May 12, 2011

(54) KITS AND PROCEDURES FOR NATURAL ORIFICE TRANSLUMENAL ENDOSCOPIC SURGERY

(75) Inventors: Ragae M. Ghabrial, Loveland, OH (US); James T. Spivey, Cincinnati, OH (US); Kurt R. Bally, Lebanon, OH (US); Duane A. Linenkugel, Cincinnati, OH (US); Gregory J. Bakos, Mason, OH (US); Kempton K. Carroll, II, Cincinnati, OH (US); Omar J. Vakharia, Cincinnati, OH (US); Robert M. Trusty, Cincinnati, OH (US)

(73) Assignee: Ethicon Endo-Surgery, Inc., Cincinnati, OH (US)

(21) Appl. No.: 12/614,143

(22) Filed: Nov. 6, 2009

(51) Int. Cl. A61B 17/94; A61B 1/00; A61B 17/06; A61B 17/295; A61B 17/32; A61B 18/14; A61B 17/10; A61B 10/04

(52) U.S. Cl. 600/564; 600/104; 606/232; 606/205; 606/170; 606/41; 606/142; 606/139

(57) ABSTRACT

Various surgical devices, kits, and/or methods are provided herein that may be useful in performing a surgical procedure through a natural orifice. Such a surgical procedure may utilize one or more devices, kits, and/or methods to create an access port to a body cavity of a patient, to perform a specific surgical procedure, and to close the access port. In various embodiments, the specific surgical procedure may comprise a sleeve gastrectomy, a ventral hernia repair, a hybrid transgastric cholecystectomy, and/or a hybrid transgastric appendectomy.

### BACKGROUND

The embodiments relate, in general, to surgical kits and procedures and, more particularly, to kits and procedures used and/or conducted through a patients natural orifice, such as the patient's mouth, anus, and/or vagina, to accomplish specific surgical goals.

Access to internal body cavities, such as the abdominal cavity, may, from time to time, be required for diagnostic and therapeutic endeavors for a variety of medical and surgical diseases. Historically, abdominal access has required a formal laparotomy or open incision to provide adequate exposure. Such procedures, which require incisions to be made in the abdomen, are not particularly well-suited for patients that may have extensive abdominal scarring from previous procedures, those persons who are morbidly obese, those individuals with abdominal wall infection, and those patients with diminished abdominal wall integrity, such as patients with burns and skin grafting. Other patients simply do not want to have a scar if it can be avoided.

Minimally invasive procedures are desirable because such procedures can reduce pain and provide relatively quick recovery times as compared with conventional open medical procedures. Many minimally invasive procedures are performed with an endoscope (including, without limitation, laparoscopes). Such procedures permit a physician to position, manipulate, and view medical instruments and accessories inside the patient through a small access opening in the patient's body. Laparoscopy is a term used to describe such an “endosurgical” approach using an endoscope (often a rigid laparoscope). In this type of procedure, accessory devices are often inserted into a patient through trocars placed through the body wall. Trocars must typically pass through several layers of overlapping tissue/muscle before reaching the abdominal cavity.

Still less invasive treatments include those that are performed through insertion of an endoscope through a natural body orifice to a treatment region. Examples of this approach include, but are not limited to, cholecystectomy, appendectomy, cystoscopy, hysteroscopy, esophagogastroduodenoscopy, and colonoscopy. Many of these procedures employ the use of a flexible endoscope during the procedure. Flexible endoscopes often have a flexible, steerable articulating section near the distal end that can be controlled by the user by utilizing controls at the proximal end. Minimally invasive therapeutic procedures to treat diseased tissue by introducing medical instruments to a tissue treatment region through a natural opening of the patient are known as Natural Orifice Translumenal Endoscopic Surgery (NOTES)™. Entry through a natural opening may further reduce the pain a patient experiences after the procedure because the tissue walls at or near the natural orifice may have less pain receptors than do the abdominal walls. Exemplary surgical procedures conducted at least partially through a natural orifice may be described in one or more of the following: U.S. Pat. No. 6,572,629 (U.S. patent application Ser. No. 09/929,125), filed Aug. 15, 2001, entitled “GASTRIC REDUCTION ENDOSCOPY” to Anthony Nicolas Kalloo et al., U.S. Pat. No. 5,297,536 (U.S. patent application Ser. No. 07/934,914), filed Aug. 25, 1992, entitled “METHOD FOR USE IN INTRA-ABDOMINAL SURGERY” to Peter J. Wilk, U.S. Pat. No. 5,458,131 (U.S. patent application Ser. No. 08/181,700), filed Jan. 14, 1994, entitled “METHOD FOR USE IN INTRA-ABDOMINAL SURGERY” to Peter J. Wilk, and/or U.S. Published Patent Application No. 2001/0049497 (U.S. patent application Ser. No. 09/815,336), filed Mar. 23, 2001, entitled “METHODS FOR DIAGNOSTIC AND THERAPEUTIC INTERVENTIONS IN THE PERITONEAL CAVITY” to Anthony Nicolas Kalloo et al., the disclosures of which are incorporated herein by reference in their respective entireties.

Some flexible endoscopes are relatively small (about 1 mm to 3 mm in diameter), and may have no integral accessory channel (also called biopsy channels or working channels). Other flexible endoscopes, including gastroscopes and colonoscopes, have integral working channels having a diameter of about 2.0 mm to about 3.7 mm for the purpose of introducing and removing medical devices and other accessory devices to perform diagnosis or therapy within the patient. As a result, the accessory devices used by a physician can be limited in size by the diameter of the accessory channel of the scope used. Additionally, the physician may be limited to a single accessory device when using the standard endoscope having one working channel.

Certain specialized endoscopes are available, such as large working channel endoscopes having a working channel of about 5-10 mm in diameter, which can be used to pass relatively large accessories, or to provide capability to suction large blood clots. Other specialized endoscopes include those having two or more working channels.

The above mentioned minimally invasive surgical procedures have changed some of the major open surgical procedures such as gall bladder removal, or a cholecystectomy, to simple outpatient surgery. Consequently, the patient's return to normal activity has changed from weeks to days. These types of surgeries are often used for repairing defects or for the removal of diseased tissue or organs from areas of the body such as the abdominal cavity.

The foregoing discussion is intended only to illustrate the present field and should not be taken as a disavowal of claim scope.

### SUMMARY

In various embodiments, a surgical kit is provided. In at least one embodiment, the surgical kit can comprise a guide system for accommodating endoscopic tools, a translumenal access device, and an expandable suture anchor. In these embodiments, the guide system can comprise a hollow overtube having a proximal end and a distal end, the distal end being substantially steerable, and an inner sheath having a proximal end and a distal end and being sized relative to the hollow overtube to permit the inner sheath to be selectively rotated and axially moved within the hollow overtube such that the distal end of the inner sheath may selectively protrude beyond the distal end of the hollow overtube. The inner sheath can also have at least one working channel formed therein and a distal end of the at least one working channel can be substantially steerable. Further, in these embodiments, the translumenal access device can comprise a catheter, an inflatable member, a hollow needle, a stylet, and a guide wire. The catheter can comprise a proximal end, a distal end, at least one first lumen, and at least one second lumen. Also, the first lumen can be configured to slidably receive a guide wire from the proximal end to the distal end of the catheter. The inflatable member can be mounted near the distal end of the catheter and in fluid communication with the second lumen. The hollow needle can be mounted on the distal end of the catheter and mounted distal to the inflatable member. The stylet can comprise a third lumen, and the stylet can be configured to be slidably disposed within the hollow needle. Also, the stylet can comprise at least one extended position and at least one retracted position. Moreover, in these embodiments, the guide wire can be slidably moveable between an extended position and a retracted position. When in the extended position, the guide wire can be extended distally from the stylet, and when in the retracted position, the guide wire can be retracted proximally from the stylet. Also, the guide wire can be configured to be received in at least a part of the first lumen and in at least a part of the third lumen.

In various embodiments, a surgical method is provided. In at least one embodiment, the method can comprise obtaining a steerable overtube comprising a body defining a lumen therethrough, wherein the body includes a distal portion and a proximal portion, placing an insertable portion of a first endoscope into the overtube's lumen, wherein the first endoscope includes at least one working channel, inserting the overtube's distal portion and the endoscope's insertable portion into a patient's natural orifice, positioning a portion of a translumenal access device through the working channel of the first endoscope, wherein the access device comprises a needle and an inflatable member mounted near the needle, puncturing a tissue wall within the patient with the needle to create an incision, locating the inflatable member within the incision, inflating the inflatable member to dilate the incision, passing the overtube's distal portion and the endoscope's insertable portion through the dilated incision, performing a specific surgical procedure within the patient, moving the overtube's distal portion and the endoscope's insertable portion out of the dilated incision, sealing the incision, and removing the steerable overtube and the first endoscope from the patient.

### BRIEF DESCRIPTION OF THE FIGURES

The novel features of the embodiments described herein are set forth with particularity in the appended claims. The embodiments, however, both as to organization and methods of operation may be better understood by reference to the following description, taken in conjunction with the accompanying drawings as follows.

FIG. 1 is a diagrammatical view illustrating a non-limiting embodiment of an endoscope inserted into an overtube and through a patient's mouth and esophagus to perform a surgical procedure.

FIG. 2 is a partial perspective view of the distal portion of the endoscope inserted through the overtube of FIG. 1.

FIG. 3 is a side view of a non-limiting embodiment of an access device extending from an endoscope inserted through an incision in a tissue wall with an inflatable member inflated within the incision.

FIG. 4A is a side view of a non-limiting embodiment of a tissue apposition device.

FIG. 4B is a side view of another non-limiting embodiment of a tissue apposition device.

FIG. 5A is a side view of a non-limiting embodiment of a flexible endoscopic translumenal overtube assembly comprising a flexible endoscope disposed within one embodiment of a flexible overtube.

FIG. 5B illustrates a steerable segment of the flexible endoscopic translumenal overtube assembly shown in FIG. 5A in an actuated state.

FIG. 6A is a side view of a non-limiting embodiment of a guide system including an overtube having a proximal end coupled to a handle assembly; a non-limiting embodiment of an inner sheath is show inserted into the overtube.

FIG. 6B is a side view of a non-limiting embodiment of the inner sheath of FIG. 6A.

FIG. 6C illustrates deployment of endoscopic instruments at a treatment site using the inner sheath of FIG. 6A.

FIG. 7 illustrates a process flowchart showing an overview of some of the steps involved before and after performing a specific surgical procedure.

FIG. 8 illustrates a process flowchart showing some of the optional surgical procedures that may be carried out as the specific surgical procedure depicted in FIG. 7.

FIG. 9 illustrates a process flowchart showing an overview of some of the steps involved when a sleeve gastrectomy procedure is the specific surgical procedure depicted in FIG. 7.

FIG. 10 illustrates a process flowchart showing an overview of some of the steps involved when a ventral hernia repair procedure is the specific surgical procedure depicted in FIG. 7.

FIG. 11 illustrates a process flowchart showing an overview of some of the steps involved when a hybrid transgastric cholecystectomy procedure is the specific surgical procedure depicted in FIG. 7.

FIG. 12 illustrates a process flowchart showing an overview of some of the steps involved when a hybrid transgastric appendectomy procedure is the specific surgical procedure depicted in FIG. 7.

FIG. 13A is a perspective view of a non-limiting embodiment of an articulating grasper in an articulated position.

FIG. 13B is an enlarged view of the articulating grasper of FIG. 13A.

FIG. 14 is a side view of a non-limiting embodiment of endoscopic scissors.

FIG. 15 is a perspective view of a non-limiting embodiment of an articulating hook knife.

FIG. 16 illustrates a non-limiting embodiment of endoscopic bipolar forceps attached to an energy source.

FIG. 17A illustrates a non-limiting embodiment of the distal portion of a flexible clip applier inserted through an overtube.

FIG. 17B illustrates the flexible clip applier and overtube of FIG. 17A with grasper devices protruding from the clip applier.

FIG. 18 is a side view of a non-limiting embodiment of an articulating needle knife.

FIG. 19 illustrates a non-limiting embodiment of an articulating specimen bag.

FIG. 20 illustrates a non-limiting embodiment of an endoscopic Maryland dissector.

FIG. 21 illustrates a non-limiting embodiment of an articulating specimen bag in position to receive a gall bladder therein.

FIG. 22A illustrates a non-limiting embodiment of a non-articulating grasper.

FIG. 22B illustrates the non-articulating grasper of FIG. 22A extending from a working channel of an endoscope.

FIG. 23 is a side view of a non-limiting embodiment of an articulating snare loop.

### DETAILED DESCRIPTION

Certain embodiments will now be described to provide an overall understanding of the principles of the structure, function, manufacture, and use of the devices and methods disclosed herein. One or more examples of these embodiments are illustrated in the accompanying drawings. Those of ordinary skill in the art will understand that the devices and methods specifically described herein and illustrated in the accompanying drawings are non-limiting embodiments and that the scope of these embodiments is defined solely by the claims. The features illustrated or described in connection with one embodiment may be combined with the features of other embodiments. Further, where an ordering of steps in a process is indicated, such ordering may be rearranged or the steps may be carried out contemporaneously as desired unless illogical or the listed order is explicitly required. Such modifications and variations are intended to be included within the scope of the appended claims.

In the following description, like reference characters designate like or corresponding parts throughout the several views. Also in the following description, it is to be understood that terms such as “forward,” “rearward,” “front,” “back,” “right,” “left,” “upwardly,” “downwardly,” “proximally,” “distally,” and the like are words of convenience and are not to be construed as limiting terms. The description below is for the purpose of describing various embodiments and is not intended to limit the appended claims.

The various embodiments generally relate to various kits, systems, and or methods for use in connection with endoscopes, including laparoscopes, for performing a surgical procedure or procedures within a patient's body cavity. The terms “endoscopic tools” and “endoscopic surgical instruments” as used herein may comprise, for example, endoscopes, lights, insufflation devices, cleaning devices, suction devices, hole-forming devices, imaging devices, cameras, graspers, clip appliers, loops, Radio Frequency (RF) ablation devices, harmonic ablation devices, scissors, knives, suturing devices, etc. However, such term is not limited to those specific devices. As the present Description proceeds, those of ordinary skill in the art will appreciate that the unique and novel features of the various instruments and methods for use thereof may be effectively employed to perform surgical procedures by inserting such endoscopic tools through a natural body lumen (mouth, anus, vagina) and/or through a transcutaneous port (abdominal trocar, cardiothoracic port) to perform surgical procedures within a body cavity.

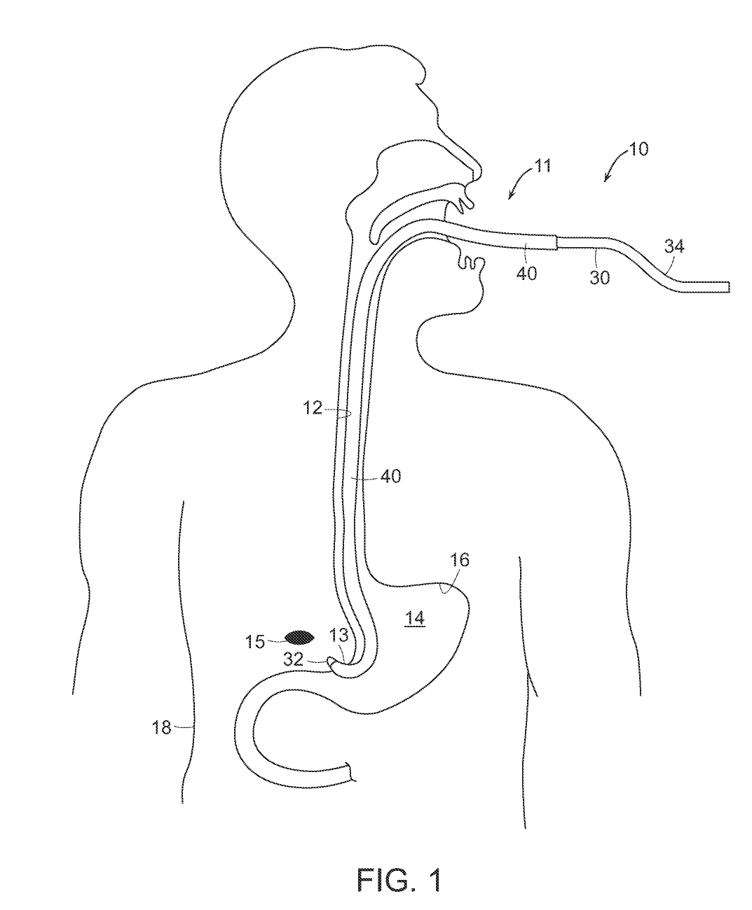

The various embodiments described herein are directed to medical devices and, more particularly, to methods and devices which can be useful in minimally invasive endoscopic procedures carried out with an endoscope and/or a similar surgical instrument. Various embodiments can include methods and devices useful during various medical procedures including, without limitation, methods and devices useful with endoscopes and methods and devices employed through naturally occurring body orifices. Accordingly, the various embodiments can include devices, systems, and/or methods useful in natural orifice translumenal endoscopic surgery (“NOTES”) procedures. As noted above, NOTES procedures may be performed transorally, transgastrically, and/or transvaginally. In at least one such embodiment, and referring now to FIG. 1, a surgical system 10, comprising an endoscope 30 and an overtube 40 is shown with the endoscope 30 inserted into the overtube 40 and inserted through a patient's mouth 11 and esophagus 12 to perform a surgical procedure on a surgical target 15, such as to remove the patient's gall bladder, or perform a cholecystectomy, for example. Additional exemplary surgical targets and/or procedures are explained in more detail below. In various embodiments, overtube 40 and/or endoscope 30 can be inserted through any suitable natural orifice in the patient to form an opening in an organ, or a portion of an organ, such as stomach wall 16, for example. The insertion of the overtube 40 and/or endoscope 30 into the patient may occur transorally (as depicted in FIG. 1), transanally, and/or transvaginally, for example. In the example depicted in FIG. 1, the overtube 40 and endoscope 30 are inserted through the mouth 11 and esophagus 12 of the patient and into the stomach 14 to form an opening 13 through the stomach wall 16.

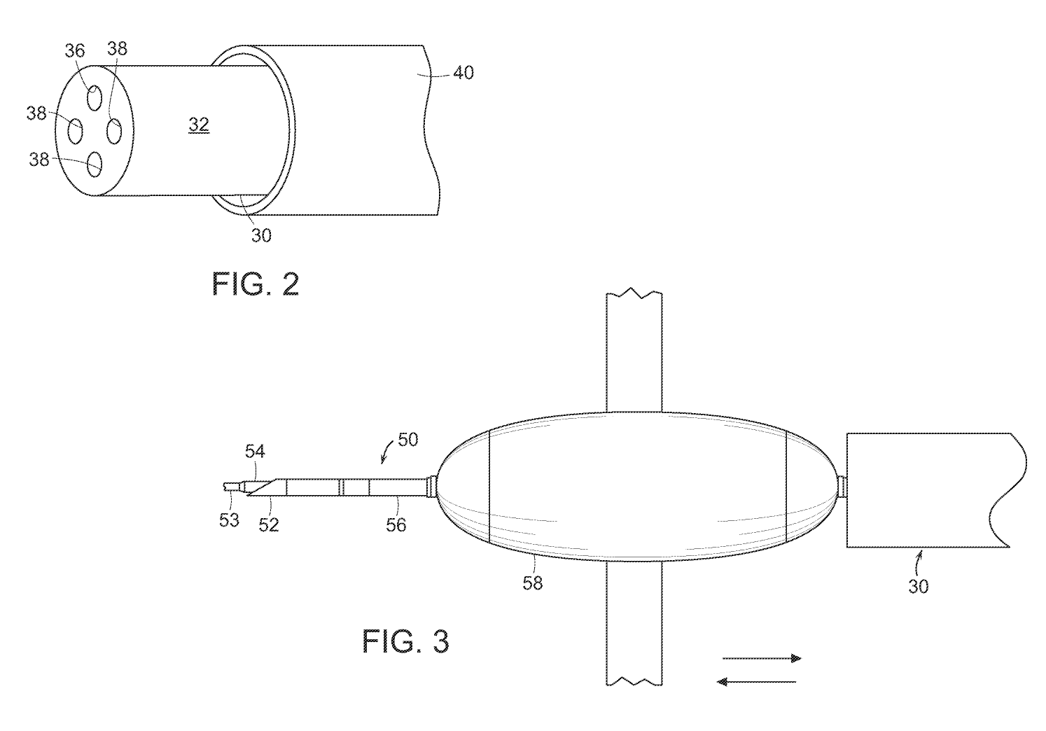

FIG. 2 is a partial perspective view of the distal portion 32 of the flexible endoscope 30 inserted through the overtube 40 of FIG. 1. A variety of different types of endoscopes are known. An exemplary, but non-limiting, endoscope and endoscopic system is illustrated and described in U.S. patent application Ser. No. 11/386,861 to Maseda, et al., entitled ENDOSCOPE WORKING CHANNEL WITH MULTIPLE FUNCTIONALITY, the disclosure of which is hereby incorporated by reference in its entirety. In various embodiments, the flexible endoscope 30 has a distal end 32 and a proximal end 34 and may operably support a video camera 36 that communicates with a video display unit that can be viewed by the surgeon during the operation. The flexible endoscope 30 may also comprise one or more working channels 38 extending therethrough for receiving various types of surgical instruments, wherein the working channels 38 may be accessed via working channel ports (not shown) of the endoscope 30.

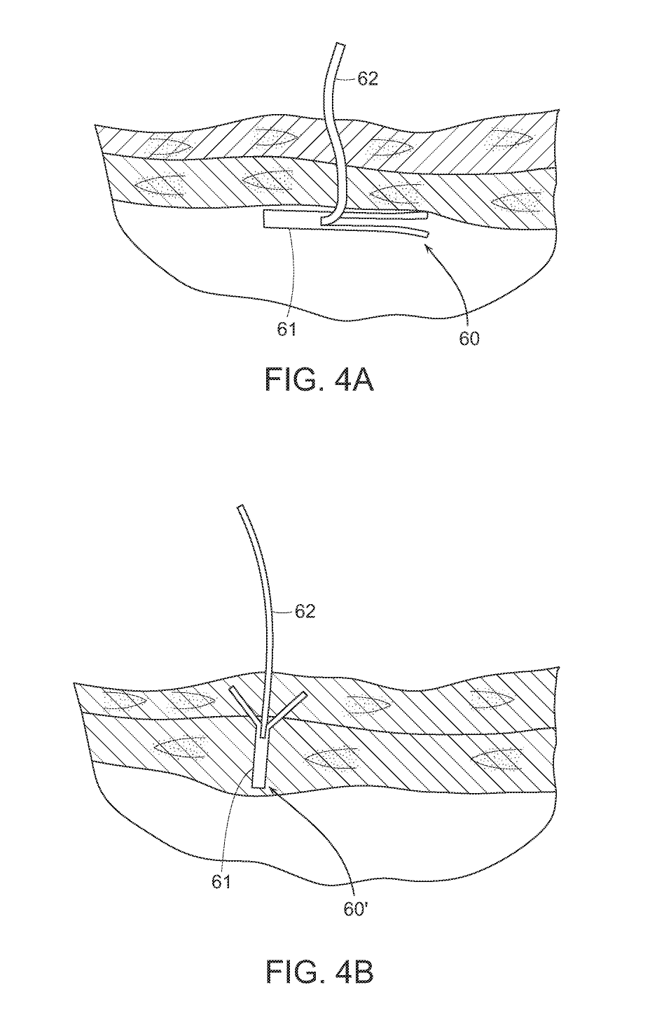

In at least one embodiment, various surgical tools and/or kit(s) are provided for performing one or more surgical procedures. Such a surgical kit may include various devices to guide surgical tools through a patient's natural orifice, gain access to a body cavity through the natural orifice, and seal an incision made within the patient's body, at or near the conclusion of the surgical procedure(s). For example, in various embodiments and referring to FIGS. 1-4A, a surgical kit may comprise one or more of a flexible trocar or overtube 40 (see FIGS. 1-2), an access device 50 (see FIG. 3), and a tissue apposition device 60 (see FIG. 4). As used herein, a surgical kit may also comprise an enclosure, such as bag or container, to hold the instrument or instruments of the kit.

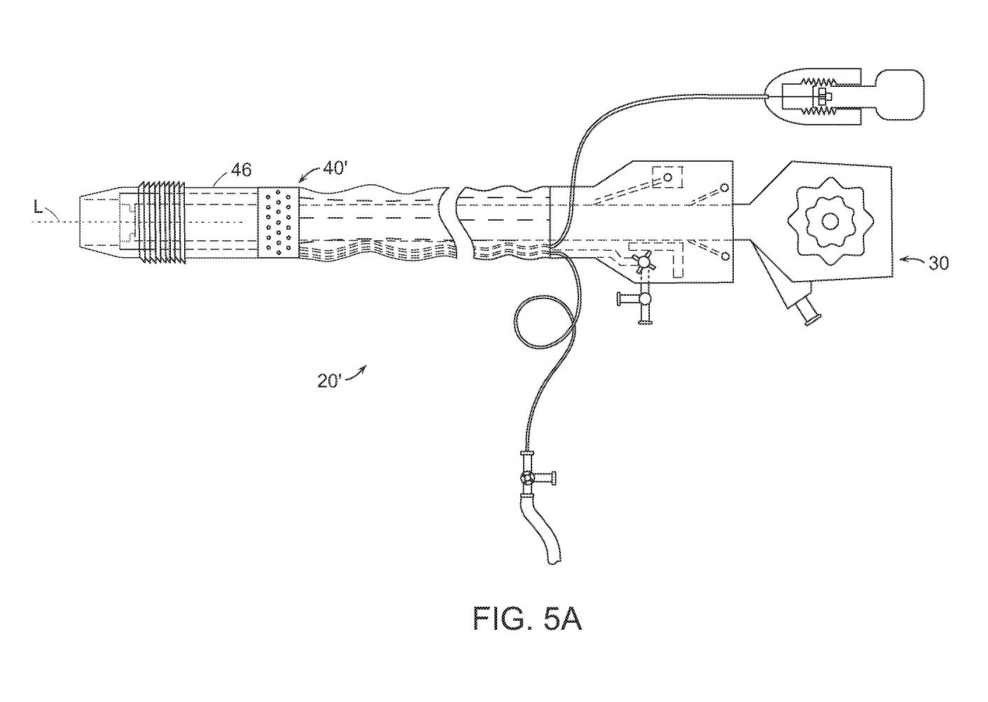

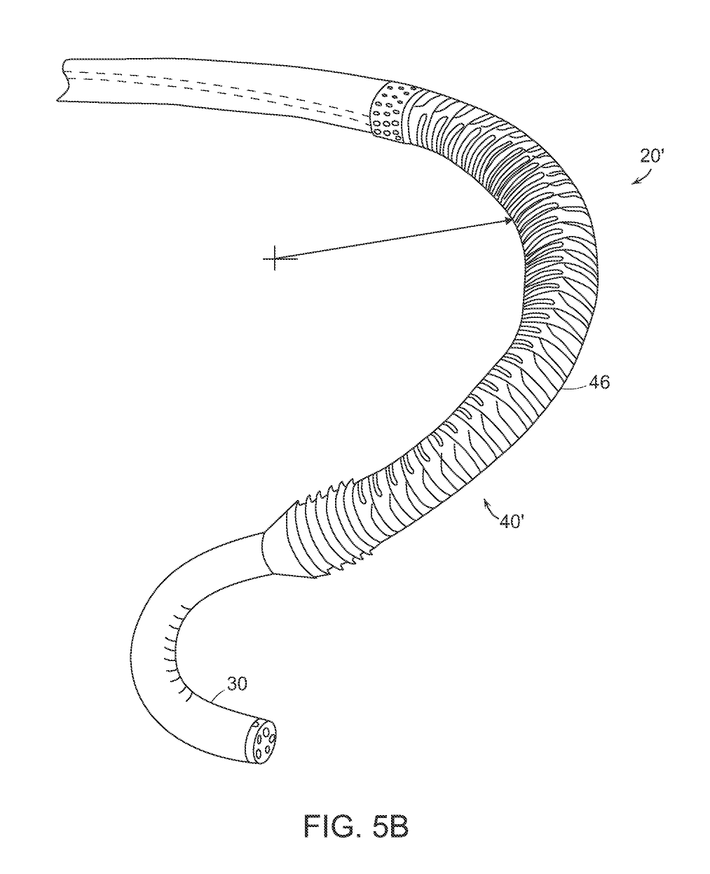

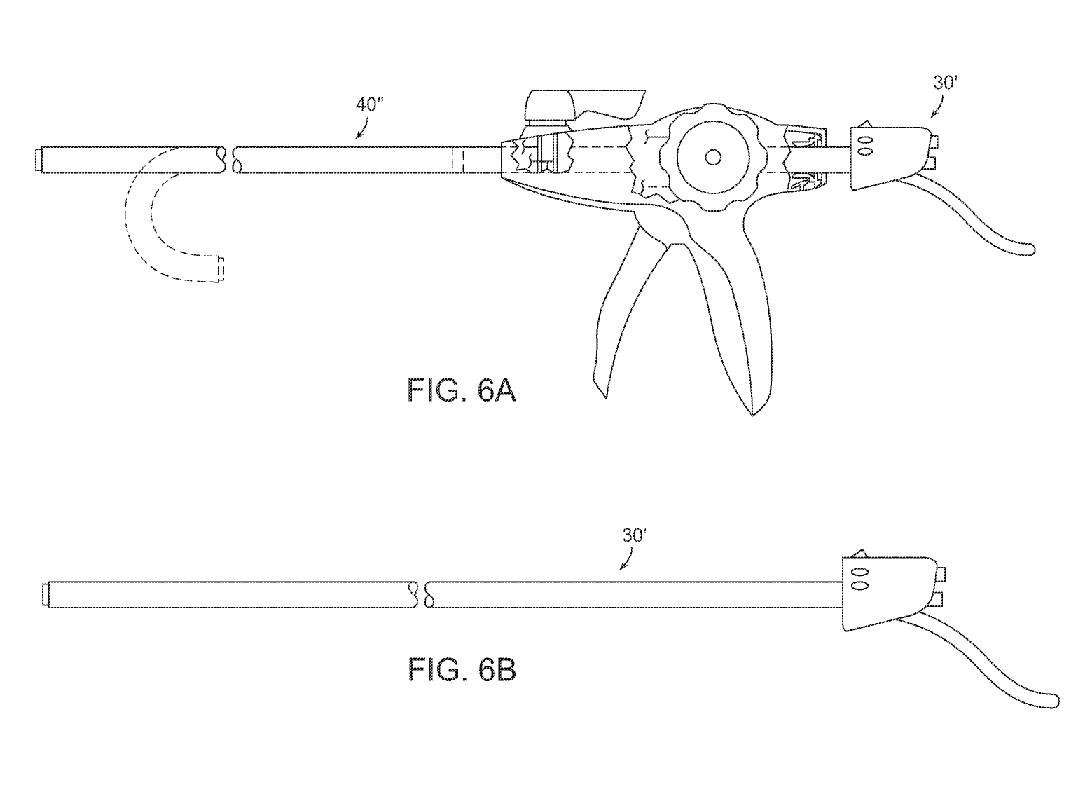

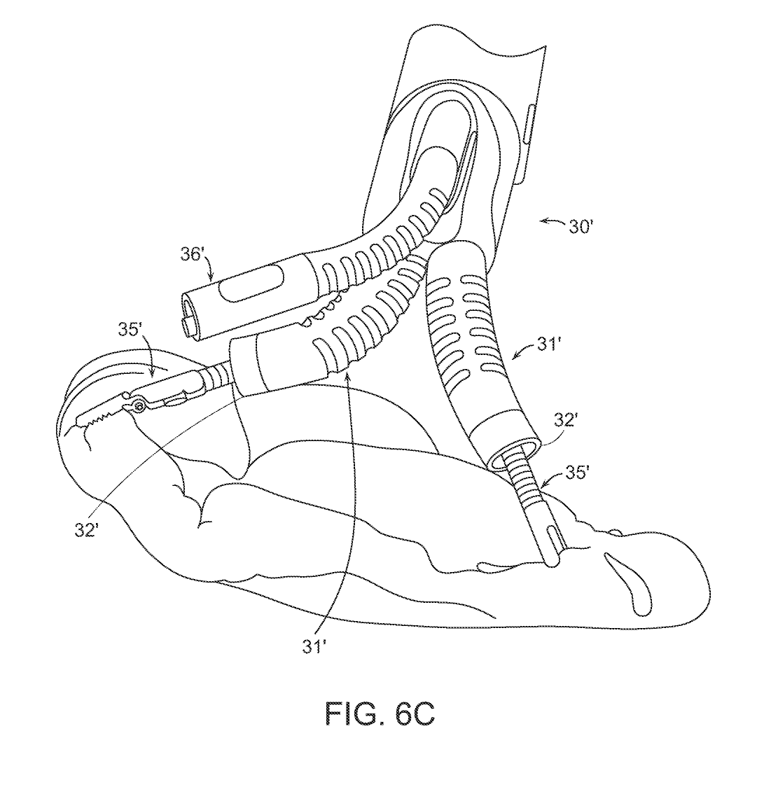

Generally, the overtube 40 may be steerable and may comprise a body defining a lumen therethrough. Exemplary overtubes are shown in FIGS. 5A-6A. FIG. 5A is a side view of one embodiment of a flexible endoscopic translumenal overtube assembly 20′ comprising a flexible endoscope 30 disposed within one embodiment of a flexible overtube 40′. FIG. 5B illustrates a steerable segment 46 of the flexible endoscopic translumenal overtube assembly 20′ shown in FIG. 5A in an actuated state. Additional details regarding overtube assembly 20′, overtube 40′, and/or similar devices may be found in U.S. patent application Ser. No. 12/172,782, filed Jul. 14, 2008, entitled “ENDOSCOPIC TRANSLUMENAL ARTICULATABLE STEERABLE OVERTUBE” to Gregory J. Bakos et al., the disclosure of which is incorporated herein by reference in its entirety.

FIG. 6A illustrates another embodiment of a guide system including an outer sheath or overtube 40″ having a proximal end coupled to a handle assembly. The overtube 40″ may be articulated or steered by various controls on the handle. Also, the overtube 40″ may accommodate an endoscope or an endoscope substitute, such as inner sheath 30′ seen in FIGS. 6A-6B. Accordingly, as used herein, the term “endoscope” includes such devices as inner sheath 30′ that may provide a working channel for an endoscopic instrument and/or a camera for visualizing inside a patient's body. Further, inner sheath 30′ may be a part of a surgical kit as well. Additional details regarding overtube 40″, inner sheath 30′, its use with overtube 40″, and/or similar devices may be found in U.S. patent application Ser. Nos. 11/894,358, filed Aug. 21, 2007, and/or 12/468,462, each entitled “MANIPULATABLE GUIDE SYSTEM AND METHODS FOR NATURAL ORIFICE TRANSLUMENAL ENDOSCOPIC SURGERY” to Robert M. Trusty, the disclosures of which are incorporated herein by reference in their respective entireties.

In such embodiments, referring still to FIGS. 6A-6B, for example, the overtube 40″ may be part of a guide system for accommodating endoscopic tools. The guide system may comprise a hollow outer sheath or overtube 40″ and an inner sheath 30′. The hollow outer sheath 40″ may have a proximal end and a distal end and the distal end may be substantially steerable. The inner sheath 30′ may also have a proximal end, a distal end, and be sized relative to the hollow outer sheath to permit the inner sheath to be selectively rotated and axially moved within the hollow outer sheath 40″ such that the distal end of the inner sheath 30′ may selectively protrude beyond the distal end of the hollow outer sheath 40″. Further, referring to FIG. 6C, for example, the inner sheath 30′ may have at least one working channel 31′ formed therein. A distal end 32′ of each working channel 31′ may be substantially steerable and may be configured to guide the tip of a flexible endoscopic device 35′ within the working channel. For instance, FIG. 6C illustrates deployment of endoscopic instruments 35′ at a treatment site from working channel distal ends 32′ that have been steered apart from each other such that the endoscopic instruments 35′ may manipulate tissue. A camera 36′ is also shown steered or articulated independently of the working channels 31′. The endoscopic instruments 35′ may include the exemplary graspers or forceps as shown or any of the other endoscopic tools described herein.

Alternatively, referring back to FIGS. 1-2, the overtube 40 may comprise a steerable overtube of a type disclosed in U.S. patent application Ser. No. 11/981,134, filed Oct. 31, 2007, entitled “ENDOSCOPIC OVERTUBES” to Gregory J. Bakos et al., the disclosure of which is herein incorporated by reference in its entirety. Additional steerable sheaths, overtubes, and/or tube arrangements may also be found in, for example, U.S. patent application Ser. No. 11/762,855, filed Jun. 14, 2007, entitled “CONTROL MECHANISM FOR FLEXIBLE ENDOSCOPE DEVICE AND METHOD OF USE” to James T. Spivey and Omar J. Vakharia, and U.S. Pat. No. 5,325,845, issued Jul. 5, 1994, to Aidar the disclosures of which are herein incorporated by reference in their respective entireties. In other embodiments, a non-steerable overtube could conceivably be employed, depending upon the application. Further details regarding overtubes or flexible trocars and their application may also be found in one or more of the following applications: U.S. patent application Ser. No. 11/382,173, filed May 8, 2006, entitled “ENDOSCOPIC TRANSLUMENAL SURGICAL SYSTEMS” to Michael S. Cropper et al.; U.S. patent application Ser. No. 11/382,182, filed May 8, 2006, also entitled “ENDOSCOPIC TRANSLUMENAL SURGICAL SYSTEMS” to Gregory J. Bakos et al.; U.S. patent application Ser. No. 11/382,196, filed May 8, 2006, also entitled “ENDOSCOPIC TRANSLUMENAL SURGICAL SYSTEMS” to Andrew Zwolinski et al.; U.S. patent application Ser. No. 11/775,477, filed Jul. 10, 2007, also entitled “ENDOSCOPIC TRANSLUMENAL SURGICAL SYSTEMS” to John P. Measamer et al.; and U.S. patent application Ser. No. 12/243,334, filed Oct. 1, 2008, also entitled “OVERTUBE WITH EXPANDABLE TIP” to Omar J. Vakharia, the disclosures of which are incorporated herein by reference in their respective entireties.

Focusing now on the access device 50, see FIG. 3, the access device 50 may comprise a needle 52 and an inflatable member 58 mounted near the needle. Additionally, the needle may be hollow and the access device may further comprise a stylet 54 slidably disposed within the needle. Further, the access device 50 may be sized and configured to fit through a working channel of an endoscope 30 such that the needle, stylet, and/or inflatable member may protrude therefrom, within the patient's body. Additional details regarding such a translumenal access device 50 and other embodiments may be found in U.S. patent application Ser. No. 12/197,653, filed Aug. 25, 2008, entitled “ENDOSCOPIC NEEDLE FOR NATURAL ORIFICE TRANSLUMENAL ENDOSCOPIC SURGERY” to Gregory J. Bakos, the disclosure of which is incorporated herein by reference in its entirety.

In such embodiments, referring still to FIG. 3, for example, a translumenal access device may comprise a catheter 56, a hollow needle 52, a stylet 54, and a guide wire 53. The catheter 56 may comprise a proximal end, a distal end, at least one first lumen, and at least one second lumen. The first lumen may be configured to slidably receive guide wire 53 from the proximal end to the distal end of the catheter 56. The inflatable member 58 may be mounted near the distal end of the catheter and be in fluid communication with the second lumen. The hollow needle 52 may be mounted on the distal end of the catheter and mounted distal to the inflatable member. The stylet 54 may comprise a third lumen. Further, the stylet 54 may be configured to be slidably disposed within the hollow needle 52 such that the stylet 54 comprises at least one extended position and at least one retracted position. The guide wire may be slidably moveable between an extended position and a retracted position. In the extended position, the guide wire may be extended distally from the stylet, and in the retracted position, the guide wire may be retracted proximally from the stylet. Further, the guide wire may be configured to be received in at least a part of the first lumen and at least a part of the third lumen.

Alternatively, the access device may be of a type described in one or more of the following applications: U.S. patent application Ser. No. 12/122,031, filed May 16, 2008, entitled “ENDOSCOPIC ROTARY ACCESS NEEDLE” to Gregory J. Bakos et al.; U.S. patent application Ser. No. 11/381,016, filed May 1, 2006, entitled “INTEGRATED GUIDEWIRE NEEDLE KNIFE DEVICE” to Gregory J. Bakos et al.; U.S. patent application Ser. No. 11/380,958, filed May 1, 2006, entitled “FLEXIBLE ENDOSCOPE SAFETY NEEDLE” to Sean P. Conlon et al., the disclosures of which are incorporated herein by reference in their respective entireties.

Moving now to the tissue apposition device 60, see FIG. 4A, the tissue apposition device may be configured to close an incision inside a patient's body. For example, the tissue apposition device 60 may include at least one suture 62 and at least one suture anchor 61 connected to the suture 62. In one embodiment, the suture anchor may be a T-tag, for example. Additionally, in at least one embodiment, the suture anchor may be expandable as shown with suture anchor 60 (FIG. 4A) and/or suture anchor 60′ (FIG. 4B), which is similar to anchor 60. Exemplary suture anchors and other embodiments may be found in U.S. patent application Ser. No. 11/274,358, filed Nov. 15, 2005, entitled “EXPANDABLE SUTURE ANCHOR” to Sean P. Conlon, the disclosure of which is incorporated herein by reference in its entirety. Further details regarding tissue apposition devices and their application may be found in one or more of the following applications: U.S. patent application Ser. No. 11/274,354, filed Nov. 15, 2005, entitled “SUTURE ANCHOR APPLICATOR” to Sean P. Conlon et al.; U.S. patent application Ser. No. 11/437,864, filed May 19, 2006, entitled “COMBINATION KNOTTING ELEMENT AND SUTURE ANCHOR APPLICATOR” to Sean P. Conlon et al.; and U.S. patent application Ser. No. 11/274,352, filed Nov. 15, 2005, entitled “SELF-SHIELDING SUTURE ANCHOR” to Sean P. Conlon et al., the disclosures of which are incorporated herein by reference in their respective entireties.

In such embodiments, referring to FIGS. 4A-4B, for example, an expandable tissue apposition device may comprise a suture anchor body 61, 61′ and a suture 62 connected to the body 61, 61′. The body may define a longitudinal axis and a bifurcated portion, with the bifurcated portion defining at least two legs, each of the legs including a first portion that is generally parallel with the longitudinal axis and a second portion that is splayed at an angle relative to the longitudinal axis. Further, the suture may extend at least partially through the bifurcated portion.

Thus, various embodiments are provided herein for devices of a surgical kit. Additional devices may also be included in such a kit. For example, in at least one embodiment, the surgical kit may further comprise an endoscope including a working channel. In such embodiments, the overtube's lumen may be sized and configured to receive at least a portion of the endoscope. The working channel may also be sized and configured to receive at least a portion of the access device. More devices that may be included in a surgical kit are described in greater detail below.

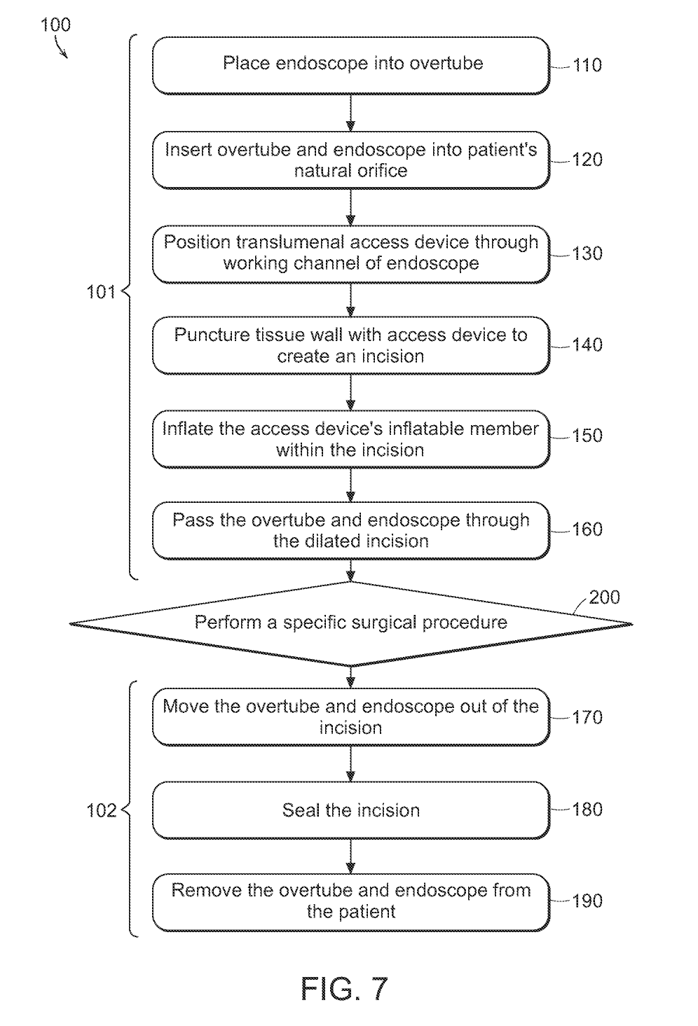

Referring now to FIG. 7, the above-discussed devices may be used to perform at least part of a surgical procedure 100 on a patient. As noted above, when performing a surgical procedure through a natural orifice, such a NOTES procedure may require access to an internal body cavity and, later, closure of the access point after the procedure is completed. FIG. 7 illustrates a process flowchart showing an overview of some of the steps of a surgical procedure 100. The surgical procedure 100 may include various steps carried out before and after performing a specific surgical procedure 200. The steps shown in FIG. 7, leading up to the specific surgical procedure 200, may be termed an access procedure 101 and the steps shown in FIG. 7, following after the specific surgical procedure 200, may be termed a closure procedure 102. Accordingly, the surgical procedure 100 may comprise access procedure 101, specific surgical procedure 200, and closure procedure 102. The details of procedures 101, 200, and 102 are explained in more depth below. Further, as described herein, details regarding any of the surgical tools mentioned below may be found in one or more of the embodiments discussed above.

Referring still to FIG. 7, in at least one embodiment, the steps of the access procedure 101 may include the following. First, an endoscope may be placed into an overtube 110. In such embodiments, the overtube may be steerable and may comprise a body defining a lumen therethrough, as described above. The overtube's body may also include a distal portion and a proximal portion. Also, an insertable portion of the endoscope may then be placed into the overtube's lumen. In other words, the overtube may be backloaded onto the endoscope, outside the patient. In more detail, and by way of example, to perform a minimally invasive procedure through a natural body opening, a physician may obtain a flexible and/or steerable trocar or overtube, a through-the-scope access device, and an endoscope also including a steerable section. The overtube may be configured to steer, retroflex, or bend in a particular direction and may also be tailored to a particular procedure. For example, transvaginal or transcolonic designs may include a mild arc, and may be shorter than those created for transgastric access. In any event, the endoscope may then be lubricated and inserted into the overtube outside the body so that the steerable section of the endoscope extends beyond the distal end of the overtube.

Second, the overtube and endsoscope, together, may be inserted into a patient's natural orifice 120. In at least one such embodiment, the overtube's distal portion and the endoscope's insertable portion may be inserted into the natural orifice. For example, the patient may be intubated and the endoscope and overtube combination may be inserted into the stomach of a patient through the mouth. Note that while portions of the present application are written from the perspective of entering the peritoneal space via a transgastric puncture in the stomach, the tools described are not limited to such an approach. Other approaches, such as transesophageal access to the thoracic cavity, transcoloninc access to the peritoneal cavity, transvaginal access to the peritoneal cavity, transvesical access to the peritoneal cavity, or transgastric access to the retroperitoneal space may also be obtained with similar steps. Further any number of body cavities and/or spaces may be accessed using such approaches, including, but not limited to the peritoneal, thoracic, retroperitoneal, and/or inguinal space of a patient.

Third, at least a portion of a translumenal access device may be positioned through a working channel of the endoscope 130. For example, the access device may be passed through the working channel of the endoscope once a target location is reached at which the physician wishes to exit the stomach. In at least one such embodiment, the physician may palpate the body wall to gain a visual cue through the view provided by the endoscope as to the proper location to exit, if desired.

Fourth, the access device may be used to puncture a tissue wall within the patient's body to create an incision 140. As discussed above, the access device may comprise a needle and an inflatable member mounted near the needle. Thus, in such embodiments, the needle may be used to puncture the tissue wall to create the incision therein. For example, the overtube may be slid past the distal end of the endoscope, slightly narrowing the view provided by the endoscope and creating a working area into which the access device may be used. Thereafter, the needle of the access device may be extended under view of the endoscope. The needle may then be pressed against the stomach's tissue wall and used to create a puncture or incision through the wall. In at least one embodiment, the needle may be rotatable to assist with creating the incision. Also, as explained above, the access device may further comprise a spring loaded stylet that also functions as a guidewire. Briefly, the stylet may be spring loaded to shield the tip of the needle from causing undesired damage to tissue. After the tissue has been pierced, the stylet may be loosened by the user and fed forward into the peritoneal space to function as a guidewire for the remainder of the access device to follow as needed.

Fifth, the inflatable member of the access device may be advanced, located, and inflated within the incision 150. For example, the needle may be first retracted back into the access device and then the balloon portion of the device may be fed into the puncture site over the guidewire. Once the deflated balloon is located in the puncture or incision in the tissue, markings on the balloon may be used to position the center portion of the balloon within the tissue, and to position the proximal portion of the balloon within the distal end of the overtube, which may include a soft, tapered shape. Accordingly, when the balloon is inflated, it may create a smooth transition from the balloon's outer surface to the overtube's outer surface. The balloon may be inflated with a liquid and/or a gas. For example, a syringe of liquid such as water or saline may be attached to a luer fitting at the proximal end of the access device and pumped into the access device to inflate the balloon. Further, a balloon inflator with a pressure gage may be used such that the balloon can be inflated to an appropriate pressure to achieve a desired external diameter. The gage may help reduce the risk of over-inflating the balloon and causing it to rupture. In any event, inflating the inflatable member within the incision may dilate the incision to a size large enough to accommodate the overtube and/or endoscope.

Sixth, the overtube and endoscope may be passed through the dilated incision 160. In at least one such embodiment, the overtube's distal portion and the endoscope's insertable portion may be passed through the dilated incision. Further, for example, once properly positioned and inflated, the access device, endoscope, and/or overtube may be moved through the dilated puncture site. In at least one such embodiment, the balloon may be held tightly against the front of the endoscope, within the overtube. Then, the user may grab both the endoscope and the overtube and advance both devices together through the dilated opening. Thereafter, the overtube's distal end may be positioned within a body cavity of the patient, such as the abdominal cavity, thereby functioning as an access site to the body cavity. The balloon may then be deflated and the access device may be removed from the working channel of the endoscope. Tubing connected to a carbon dioxide insufflator may then be attached to a stopcock on the proximal housing of the overtube to insufflate and create operative space in the peritoneal cavity. Accordingly, once the access site within the patient has been established, the endoscope may be freely passed in or out of the overtube without losing the access site or insufflation pressure.

In any event, after positioning the overtube within a body cavity of the patient, a specific surgical procedure 200 may be carried out, as explained in more detail below. In various embodiments, the desired specific surgical procedure can be performed using the endoscope. Further, different scopes or tools may be inserted to the operative site through the overtube to perform the procedure. These and other embodiments are discussed further below.

Referring still to FIG. 7, in at least one embodiment, once the specific surgical procedure 200 is completed, the closure procedure 102 may be executed. The steps of the closure procedure 102 may include the following. First, the overtube and endoscope may be moved out of the incision 170. In at least one such embodiment, the overtube's distal portion and the endoscope's insertable portion may be moved out of the dilated incision, back towards the natural orifice. For example, the endoscope and overtube can be pulled back into the stomach.

Second, the incision may be sealed 180. In various embodiments, the incision may be sealed using, for example, at least one clip, staple, endoloop, suturing device, and/or T-tag and/or by another closure technique.

Third, and finally, the overtube and endoscope may be removed from the patient 190, thereby completing the surgical procedure.

Additional steps may be added to and/or substituted for the above steps of the various procedures as desired. Further, the above steps are not intended to be comprehensive or otherwise limiting. For example, discussion is not provided about anesthetizing the patient, but it is understood that a NOTES procedure would likely include such a step. Also, as mentioned above, the ordering of the above steps may be rearranged or two or more steps may be carried out contemporaneously as desired unless illogical or the order is explicitly required.

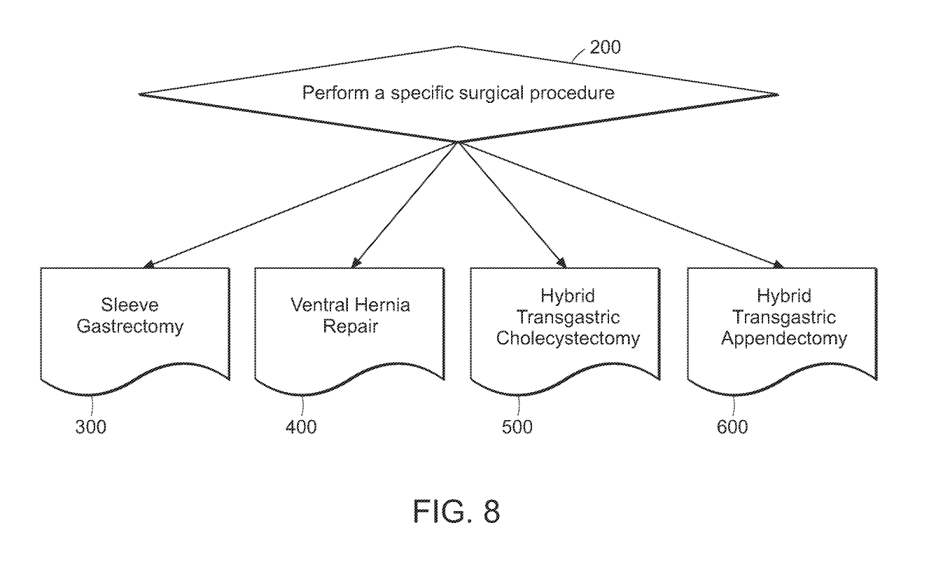

Referring still to FIG. 7, the specific surgical procedure 200 may be any of a number of surgical procedures where access to a body cavity through a natural orifice may be desirable. By way of non-limiting example, and referring now to FIG. 8, which illustrates some of the optional surgical procedures that may be carried out according to various embodiments, the specific surgical procedure 200 may include a sleeve gastrectomy 300, a ventral hernia repair 400, a hybrid transgastric cholecystectomy 500, and/or a hybrid transgastric appendectomy 600.

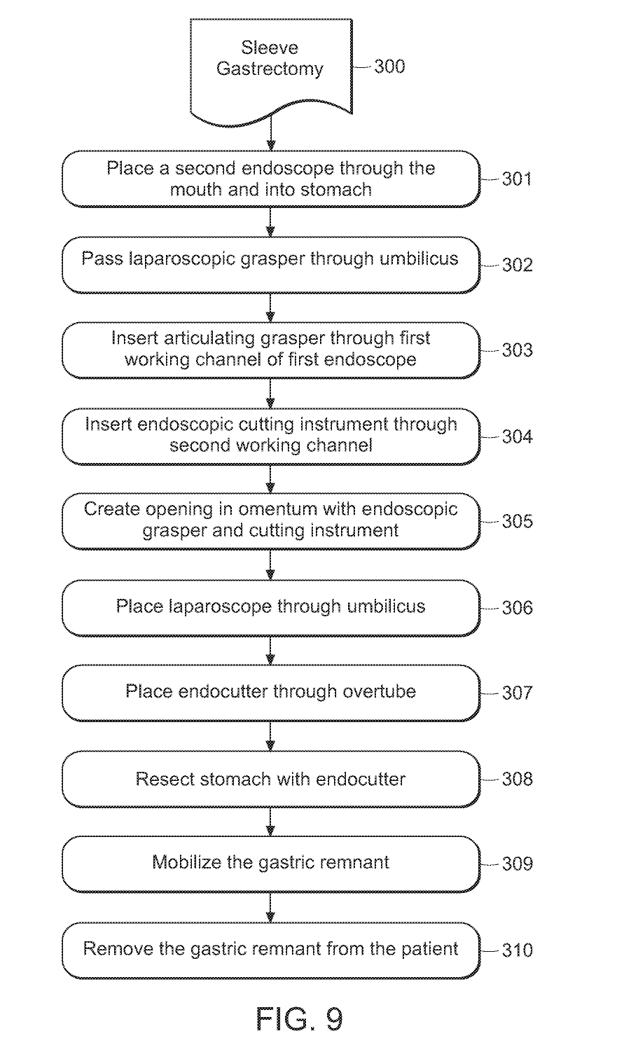

Focusing now on one exemplary embodiment, the specific surgical procedure 200 may include a sleeve gastrectomy 300, see FIGS. 8 and 9. Further to the surgical devices discussed above, additional devices which may be useful for a NOTES sleeve gastrectomy may include an articulating grasper, an endoscopic cutting instrument, and/or an endocutter configured to cut and seal tissue. One or more of these devices may also be a part of a surgical kit. Accordingly, in various embodiments, a surgical kit may include an overtube, an access device, and a tissue apposition device, as discussed above. Further, the surgical kit may also include an articulating grasper, an endoscopic cutting instrument, and an endocutter. Additional details regarding these instruments are provided below.

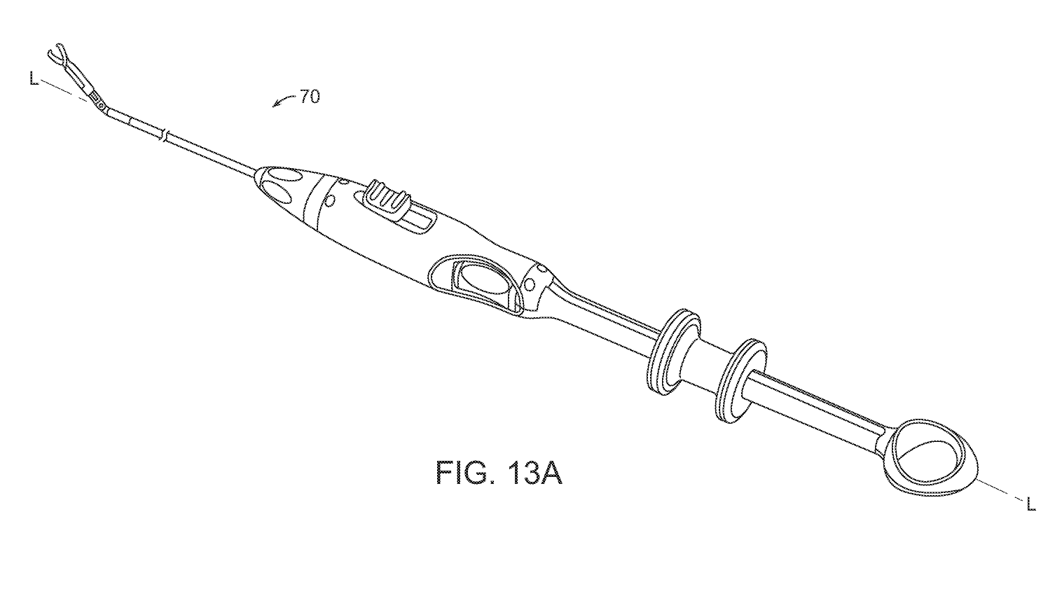

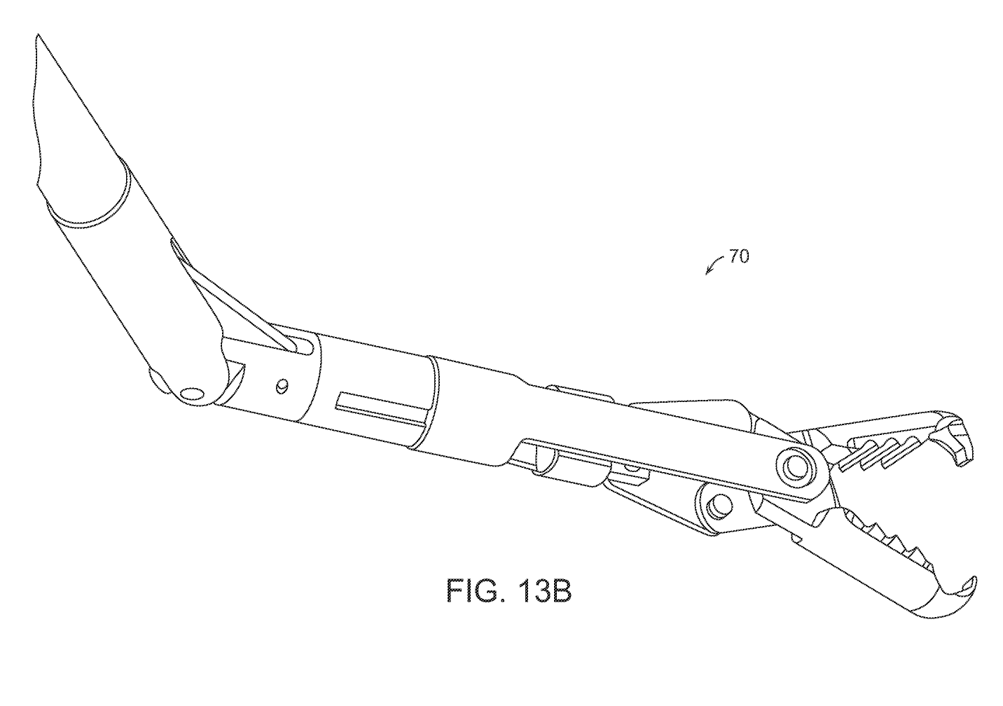

In at least one embodiment, the articulating grasper may be articulating grasper 70, which may articulate to an articulated position as shown in FIGS. 13A-13B. The articulating grasper 70 may be sized and configured to fit through a working channel of an endoscope and may be articulated once inside a patient's body cavity through the endoscope. More details regarding the articulating grasper 70 and other embodiments can be found in U.S. patent application Ser. No. 11/610,803, filed Dec. 14, 2006, entitled “MANUALLY ARTICULATING DEVICES” to Rudolph H. Nobis et al., the disclosure of which is incorporated herein by reference in its entirety. In such embodiments, the articulating grasper 70 may comprise an elongate shaft, a three-bar linkage, a grasper, and an articulation actuator. The elongate shaft may have proximal and distal ends. The three-bar linkage may also have proximal and distal ends and the proximal end may be coupled to the distal end of the elongate shaft. The grasper may be coupled to the distal end of the three-bar linkage. Further, the articulation actuator may extend through the elongate shaft and may be effective to laterally articulate the three-bar linkage relative to a longitudinal axis of the elongate shaft to angularly orient the grasper relative to the elongate shaft.

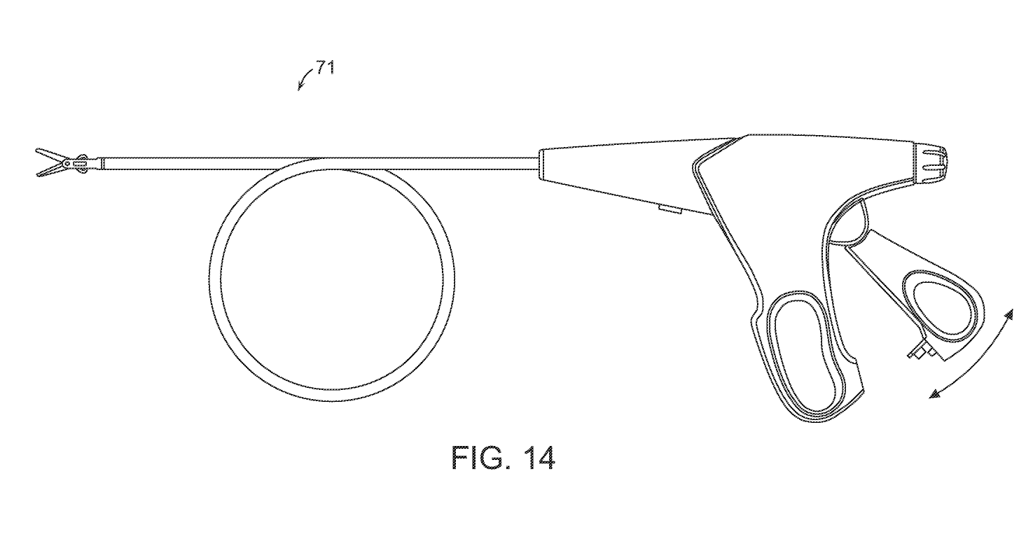

In various embodiments, the endoscopic cutting instrument may include endoscopic scissors and/or an articulating hook knife or other device configured to cut tissue. In any event, the endoscopic cutting instrument may be sized and configured to fit through a working channel of an endoscope. Referring now to FIG. 14, endoscopic scissors, such as endoscopic scissors 71 may comprise a pair of cutting blades that may be passed through an endoscope. More details regarding endoscopic scissors 71 and other embodiments can be found in U.S. patent application Ser. No. 12,364,172, filed Feb. 2, 2009, entitled “SURGICAL SCISSORS” to James T. Spivey et al., the disclosure of which is incorporated herein by reference in its entirety. In such embodiments, the endoscopic scissors 71 may comprise a clevis, a first blade member, a second blade member, a fastener, and a reciprocating shuttle, a handle, a flexible shaft, and a translating member. In more detail, the clevis may comprise a pair of arms. The first blade member may comprise a first distally positioned blade end and a first proximally positioned cam defining a first cam slot. The second blade member may comprise a second distally positioned blade and a second proximally positioned cam defining a second cam slot. Further, the fastener may be positioned to pivotably couple the first blade member and the second blade member to the clevis about a pivot point. The fastener may be held in tension by the clevis. The reciprocating shuttle may comprise at least one pin positioned within the first cam slot and the second cam slot such that distally-directed motion of the shuttle causes the first and second blade members to open and proximally-directed motion of the shuttle causes the first and second blade members to close. Also, the handle may comprise an actuator selectively positionable in a first position and a second position. The translating member may be coupled to the shuttle, extending through the flexible shaft and coupled to the actuator such that placing the actuator in the first position causes the shuttle to translate distally and placing the actuator in the second position causes the shuttle to translate proximally. Alternative embodiments of one or more endoscopic scissors may also be found in U.S. patent application Ser. No. 11/610,803, filed Dec. 14, 2006, entitled “MANUALLY ARTICULATING DEVICES” to Rudolph H. Nobis et al., noted above.

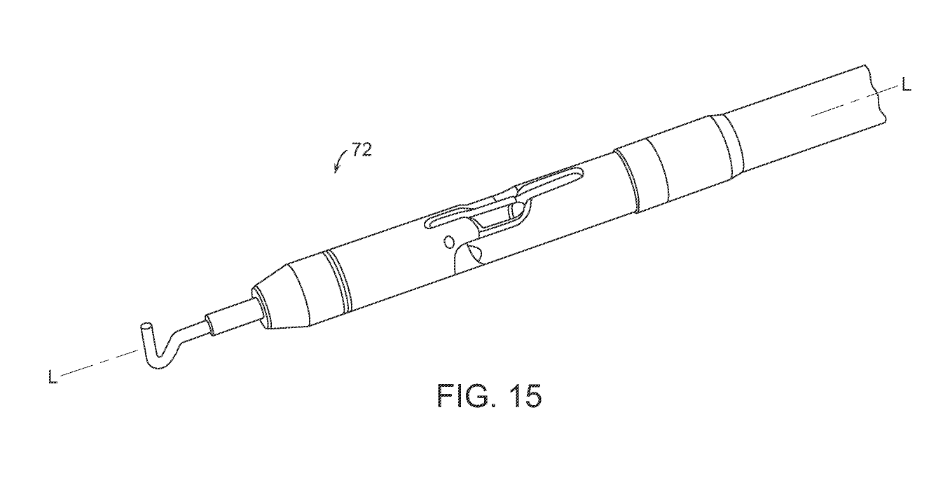

Referring now to FIG. 15, an articulating hook knife, such as articulating hook knife 72 may comprise a knife blade that may be passed through a working channel of an endoscope. More details regarding articulating hook knife 72 and other embodiments can be found in U.S. patent application Ser. No. 12/133,953, filed Jun. 5, 2008, entitled “MANUALLY ARTICULATING DEVICES” to Rudolph H. Nobis et al., the disclosure of which is incorporated herein by reference in its entirety. In such embodiments, the articulating hook knife may comprise an elongate shaft, an articulation joint, an actuation wire, and an end effector. The elongate shaft may comprise proximal and distal ends. The articulation joint may also comprise proximal and distal ends, and the proximal end of the joint may be coupled to the distal end of the elongate shaft. The actuation wire may extend through the elongate shaft and the articulation joint. Further, the end effector may comprise a distal tip coupled to the distal end of the articulation joint, and a hook knife disposed adjacent the distal tip. The distal tip may receive a distal end of the actuation wire therethrough. The hook knife may comprise proximal and distal ends, and the proximal end of the hook knife attached to the distal end of the actuation wire. Additionally, the actuation wire may be translatable along a longitudinal axis of the elongate shaft to extend and retract the distal end of the hook knife relative to the distal tip. Also, the articulation joint may be articulatable relative to the longitudinal axis of the elongate shaft to allow the end effector to be angularly oriented relative to the elongate shaft.

In various embodiments, the endocutter may be of a type found in U.S. Pat. Nos. 7,000,818 and/or 7,549,564, for example, the disclosures of which are incorporated herein by reference in their respective entireties. In such embodiments, the endocutter may comprise an elongated shaft operatively coupled to an end effector configured to cut and seal tissue, with staples, for example. In at least one embodiment, the elongated shaft may be flexible and/or sized and configured to fit through a working channel of an endoscope. Further, in such embodiments, it will be appreciated that the end effector may also be sized and configured to fit through the working channel and may also be articulated with respect to the elongated shaft. Alternatively, in at least one embodiment, the elongated shaft may be rigid and/or the end effector may be articulated with respect to the shaft. Further, endocutter may be longer than traditional endocutters to better enable a surgeon to reach far enough, with the endocutter, through a natural orifice to a body cavity. In at least on exemplary embodiment, the elongated shaft and/or the elongated shaft plus the end effector may total approximately 82 cm in length.

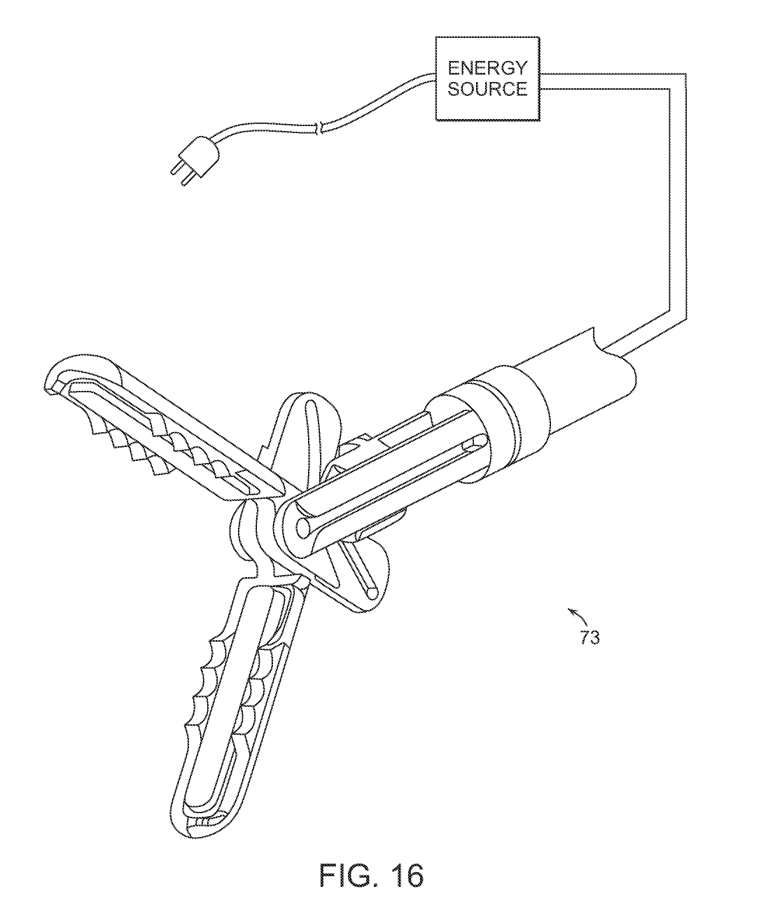

In various embodiments, the surgical kit for a sleeve gastrectomy procedure may include additional surgical instruments. For example, the surgical kit may further comprise endoscopic bipolar forceps, such as endoscopic bipolar forceps 73, see FIG. 16, that may be sized and configured to fit through a working channel of an endoscope. More details regarding endoscopic bipolar forceps 73 and other embodiments can be found in U.S. patent application Ser. No. 12/203,330, filed Sep. 3, 2008, entitled SURGICAL GRASPING DEVICE” to Matthew D. Holcomb et al., the disclosure of which is incorporated herein by reference in its entirety. In such embodiments, the endoscopic bipolar forceps 73 may comprise an elongated flexible member, a clevis, first and second jaw members, and an elongated actuator member. The elongated flexible member may have a proximal end and a distal end, and the flexible member may comprise at least one lumen. The clevis may be coupled to the elongated flexible member. Also, the first and second jaw members may be pivotally coupled to the clevis forming a clamp jaw, and the first and second jaw members may comprise respective first and second electrodes to couple to an electrical waveform generator. Further, the elongated actuator member may be slidably received within the lumen, and the elongated actuator member may be coupled to the clevis. Longitudinal motion of the elongated actuator element in a first longitudinal direction may thus open the first and second jaw members and longitudinal motion in a second opposite direction may thus close the first and second jaw members. Additionally, the first and second electrodes may be adapted to couple to an electrical waveform generator and to receive an electrical waveform sufficient to electrically ablate tissue located between the first and second jaw members. Alternative embodiments of endoscopic bipolar forceps may also be found in U.S. patent application Ser. No. 11/897,676, filed Aug. 31, 2007, entitled “ELECTRICAL ABLATION SURGICAL INSTRUMENTS” to Gary L. Long et al., and/or U.S. patent application Ser. No. 11/986,420, filed Nov. 21, 2007, entitled “BIPOLAR FORCEPS” to Ragae M. Ghabrial et al., the disclosures of which are incorporated herein by reference in their respective entireties.

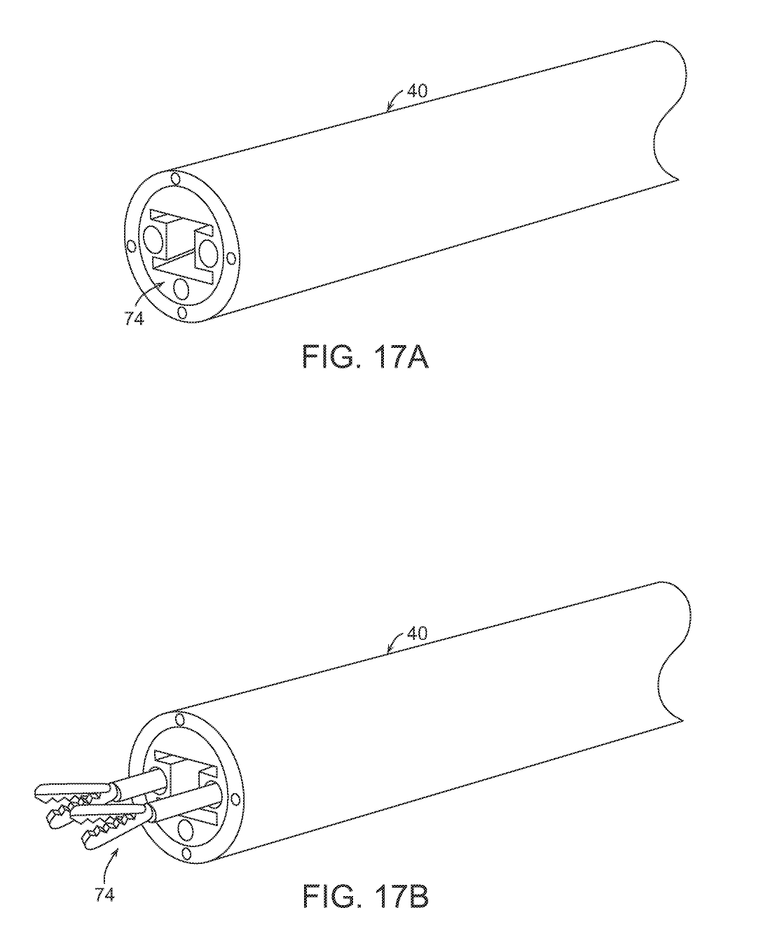

In at least one embodiment, the surgical kit for a sleeve gastrectomy may further comprise a flexible clip applier, such as flexible clip applier 74, see FIGS. 17A-17B, that may be sized and configured to fit through an overtube. More details regarding flexible clip applier 74 and other embodiments can be found in U.S. patent application Ser. No. 12/172,766, filed Jul. 14, 2008, entitled “TISSUE APPOSITION CLIP APPLICATION DEVICES AND METHODS” to Jason L. Harris et al., the disclosure of which is incorporated herein by reference in its entirety. In such embodiments, the flexible clip applier 74 may comprise an elongate clip magazine having an axial clip passage therein for receiving a plurality of tissue apposition clips therein, at least one grasper lumen in the elongate clip magazine apart from the axial clip passage and configured to movably accommodate a corresponding grasper device therethrough to manipulate tissue relative to a distal end of the elongate clip magazine, and an advancement member for applying an advancement motion to the tissue apposition clips in the axial passage to cause the tissue apposition clips to move out of the axial clip passage in seriatim. Alternative embodiments of one or more flexible clip appliers and other embodiments may also be found in U.S. patent application Ser. No. 12/170,126, entitled “DEVICES AND METHODS FOR PLACING OCCLUSION FASTENERS,” the disclosure of which is incorporated herein by reference in its entirety.

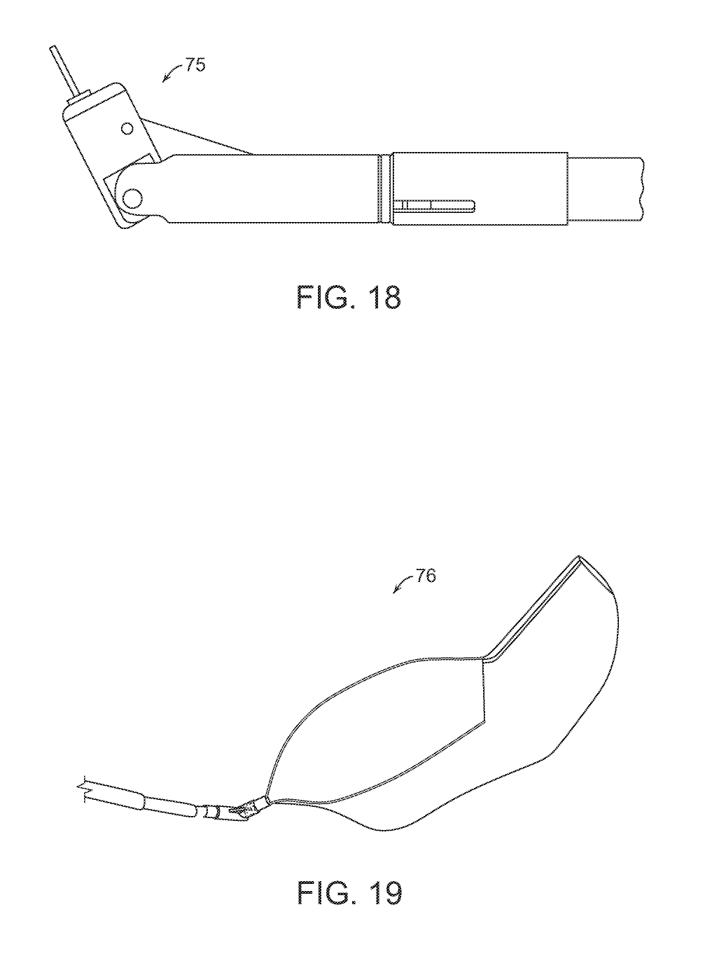

In at least one embodiment, the surgical kit for a sleeve gastrectomy may further comprise an articulating needle knife, such as articulating needle knife 75, see FIG. 18, that may be sized and configured to fit through a working channel of an endoscope. More details regarding articulating needle knife 75 and other embodiments can be found in U.S. patent application Ser. No. 11/610,803, filed Dec. 14, 2006, entitled “MANUALLY ARTICULATING DEVICES” to Rudolph H. Nobis et al., noted above. In such embodiments, the articulating needle knife 75 may comprise an elongate shaft having proximal and distal ends, a three-bar linkage having proximal and distal ends, the proximal end being coupled to the distal end of the elongate shaft, a needle knife coupled to the distal end of the three-bar linkage, and an articulation actuator extending through the elongate shaft and effective to laterally articulate the three-bar linkage relative to a longitudinal axis of the elongate shaft to angularly orient the end effector relative to the elongate shaft.

In at least one embodiment, the surgical kit for a sleeve gastrectomy may further comprise an articulating specimen bag, such as articulating specimen bag 76, see FIG. 19, that may be sized and configured to fit through an endoscope and that may be articulated and/or opened within a body cavity of a patient. More details regarding articulating specimen bag 76 and other embodiments can be found in U.S. patent application Ser. No. 12/133,109, filed Jun. 4, 2008, entitled “ENDOSCOPIC DROP OFF BAG” to Andrew M. Zwolinski et al., the disclosure of which is incorporated herein by reference in its entirety. In such embodiments, the articulating specimen bag 76 may comprise a hybrid shaft, at least one collapsible arm, a bag, a knot pusher, an articulating joint, and an outer sheath. In more detail, the hybrid shaft may have a rigid proximal end and a flexible distal end, and the hybrid shaft may extend from a proximal handle to the distal end of the articulating specimen bag. The collapsible arm may be located at the distal end of the hybrid shaft, and the bag may have an open end and a closed end. Further, the bag may be configured to be retained upon the collapsible arm. The knot pusher may be located at the distal end of the hybrid shaft and the articulating joint may connect the collapsible arm to the hybrid shaft. Also, the outer sheath may extend from a distal handle to the distal end of the articulating specimen bag. Additional embodiments of an articulating specimen bag and other embodiments may also be found in U.S. patent application Ser. No. 12/234,425, filed Sep. 19, 2008, entitled “RIGIDIZABLE SURGICAL INSTRUMENT” to Andrew M. Zwolinski et al., the disclosure of which is incorporated herein by reference in its entirety.

The above devices are just some of the surgical tools that may be part of a surgical kit used in a sleeve gastrectomy procedure. Moving now to the details of one such procedure, FIG. 9 illustrates a process flowchart showing an overview of some of the steps involved in the sleeve gastrectomy procedure 300, which may use some of the above described surgical tools. Details regarding any of the surgical tools mentioned below may be found in one or more of the embodiments discussed above. The steps leading up to those shown in FIG. 9 may include those of the access procedure 101 shown in FIG. 7, and discussed above. However, when performing such a procedure, in at least one embodiment, the natural orifice used to gain access to the patient's body cavity, discussed above, may be the colon and/or the vagina. Placing the overtube and/or first endoscope through one of these non-oral natural orifices, i.e., transvaginally and/or transcolonically, may allow a second endoscope to be introduced orally and to function as a guide for stapling the stomach, as discussed below.

In more detail, the steps of a sleeve grastrectomy procedure 300, according to at least one embodiment, may further include the following. First, the second endoscope may be placed through the patient's mouth and into the stomach 301. Second, a laparoscopic grasper may be passed through the patient's umbilicus 302. In at least one embodiment, a laparoscopic trocar and/or laparoscopic disc or hand access device, as is known in the art, may be placed at the umbilicus to create a trans-umbilicus port. Accordingly, the laparoscopic grasper may then be passed through the trans-umbilicus port to assist with the procedure.

Third, the articulating grasper may be inserted through a first working channel of the first endoscope 303. Fourth, the endoscopic cutting instrument may similarly be inserted through the second working channel of the first endoscope 304. In such embodiments, the first endoscope may include at least two working channels. In at least one embodiment, a first working channel may be approximately 3.7 mm in diameter, and a second working channel may be approximately 2.8 mm in diameter. Accordingly, the articulating grasper may be inserted through the first or 3.7 mm channel and the endoscopic cutting instrument may be inserted through the second or 2.8 mm channel.

Fifth, an opening or window in the patient's omentum may be created with the endoscopic grasper and the endoscopic cutting instrument 305. The laparoscopic grasper, operating through the umbilicus, may be used to manipulate the stomach and greater omentum to assist with this step. In any event, as noted above, the first endoscope may be positioned through the vagina or colon, through the overtube, and into the abdominal cavity. Then, using the articulating grasper and the endoscopic cutting instrument, an opening in the omentum may be created along the greater curvature of the stomach to later allow the endocutter to access the gastric serosa at the desired location. In various embodiments, the endoscopic cutting instrument may be an articulating hook knife or endoscopic scissors or another endoscopic instrument configured to cut tissue.

Sixth, a laparoscope may be placed through the patient's umbilicus 306. In at least one embodiment, the laparoscopic grasper may be removed from the umbilicus before placing the laparoscope therethrough. In any event, the laparoscope may be used to help visualize the operative site.

Seventh, the endocutter may be placed through the overtube's lumen 307. As mentioned above, an endocutter may be configured to cut and seal tissue. Accordingly, the endocutter may include a cutting member, e.g., a knife blade, and a sealing member, e.g., staples with a staple driver. Exemplary endocutters may be found in U.S. Pat. Nos. 7,000,818 and/or 7,549,564, noted above. In at least one embodiment, the endocutter may be a long, rigid linear endocutter, as discussed above. In such embodiments, the first endoscope may be removed from the overtube before introducing the endocutter. Alternatively, in at least one embodiment, the endocutter may be a flexible, endoscopic endocutter, as discussed above. In such embodiments, the first endoscope may be left positioned through the overtube to allow one of its working channels to function as a guide for the endocutter.

Eighth, a portion of the patient's stomach may be resected with the endocutter 308 to form a gastric remnant. In at least one embodiment, as mentioned above, the second endoscope may provide a guide for the endocutter. An optical dilator may be used during this step to facilitate stapler guidance. In at least one embodiment the optical dilator may be of a type described in one or more of the following applications, each entitled “ENDOSCOPIC TRANSLUMENAL SURGICAL SYSTEMS”: U.S. patent application Ser. No. 11/382,173, filed May 8, 2006, to Michael S. Cropper et al.; U.S. patent application Ser. No. 11/382,182, filed May 8, 2006, to Gregory J. Bakos et al.; U.S. patent application Ser. No. 11/382,196, filed May 8, 2006, to Andrew Zwolinski et al.; and U.S. patent application Ser. No. 11/775,477, filed Jul. 10, 2007, to John P. Measamer et al. In any event, the endocutter may be used to resect the stomach from the pyloric antrum cephalad to the cardiac zone. In at least one embodiment, a second laparoscopic port may be passed through the patient's abdominal wall to (re)introduce a laparoscopic grasper to further manipulate the stomach during the resection.

Ninth, the gastric remnant may be mobilized from the patient's vasculature with the endocutter 309. In more detail and in at least one embodiment, the endocutter may be used to divide the greater curvature vascular arcade up to and including the short gastric vessels. Alternatively, in at least one embodiment, endoscopic bipolar forceps or a ligating, flexible clip applier can be used in conjunction with endoscopic flexible scissors and/or an articulating hook knife to accomplish this step. These devices are discussed in more detail below. In any event, resecting the stomach prior to mobilization may help during retraction for a NOTES sleeve gastrectomy procedure. Resecting the stomach first may help keep the greater curve of the stomach out of the way during resection. However, the current laparoscopic standard procedure is to mobilize the stomach prior to resection. Accordingly, in at least one embodiment, the stomach may be mobilized prior to resection.

Tenth, the gastric remnant may be removed from the patient 310. Various options may be utilized to accomplish this step. In one embodiment, the vaginal or colonic opening may be enlarged using, for example, an endoscopic needle knife, the endoscopic flexible scissors, and/or the articulating hook knife. Then, the overtube or flexible trocar may be reintroduced into the enlarged opening. Next, the laparoscopic grasper may be inserted through the overtube and used to grasp the gastric remnant. Finally, the gastric remnant and the overtube may be removed under laparoscopic visual guidance.

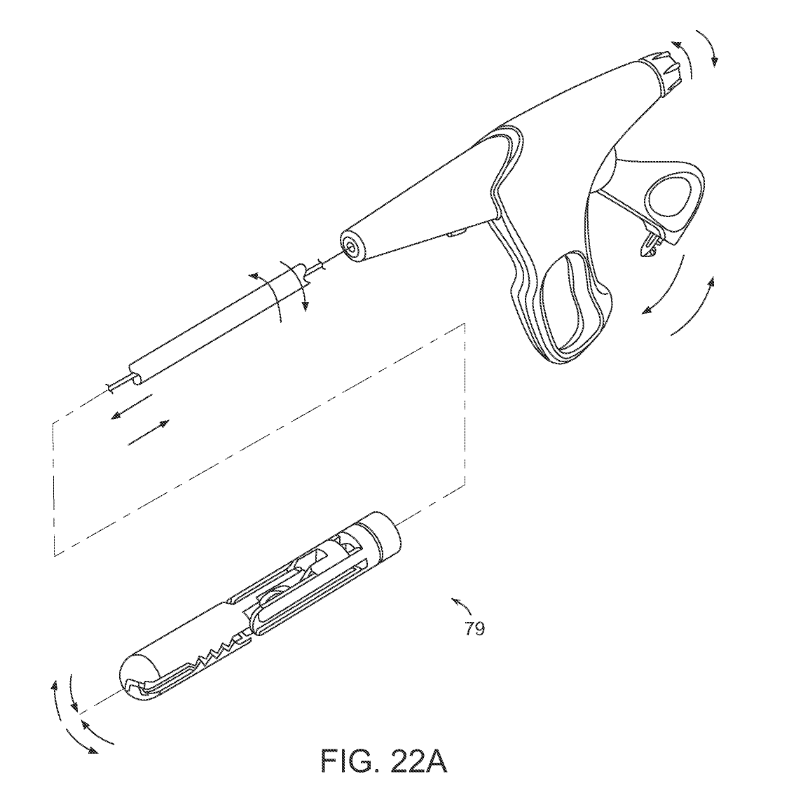

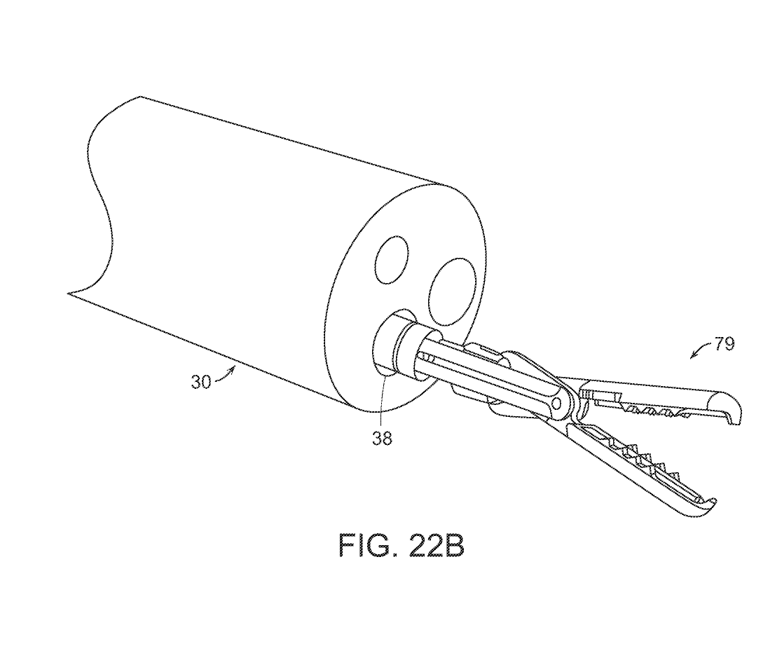

In another embodiment, the gastric remnant may be divided into small pieces under laparoscopic visual guidance using the endocutter through the overtube. Then, the gastric remnant pieces and the overtube may be removed as described above. Alternatively, the gastric remnant pieces may be removed using endoscopic graspers and one or more articulating specimen bags. Endoscopic graspers may be articulating, such as articulating grasper 70 discussed above and seen in FIGS. 13A-13B, or non-articulating. An exemplary non-articulating grasper 79 can be seen in FIGS. 22A-22B. FIG. 22A illustrates the non-articulating grasper 79 including actuation and rotational controls. As with other endoscopic tools described herein, the non-articulating grasper 79 may be sized and configured to fit through a working channel of an endoscope. For example, FIG. 22B illustrates the non-articulating grasper 79 extending from a working channel 38 of an endoscope 30. Additional details regarding non-articulating grasper 79 and other embodiments may be found in U.S. patent application Ser. No. 12/203,330, filed Sep. 3, 2008, entitled SURGICAL GRASPING DEVICE” to Matthew D. Holcomb et al., noted above.

In yet another embodiment, the laparoscopic port site may be enlarged and the gastric remnant and/or remnant pieces, if so divided, may be removed therethrough. A laparoscopic specimen bag may be used for this step. Further, in still another embodiment, if the laparoscopic disc or hand access device is used, as discussed above, the gastric remnant may be removed through the disc with a user's hand or a laparoscopic grasper.

After completing the sleeve gastrectomy procedure 300, the closure procedure 102 outlined in FIG. 7, and discussed above, may be executed to seal the incision and remove the surgical tools from the patient. Also, as mentioned above, the ordering of the above steps may be rearranged or two or more steps may be carried out contemporaneously as desired unless illogical or the order is explicitly required.

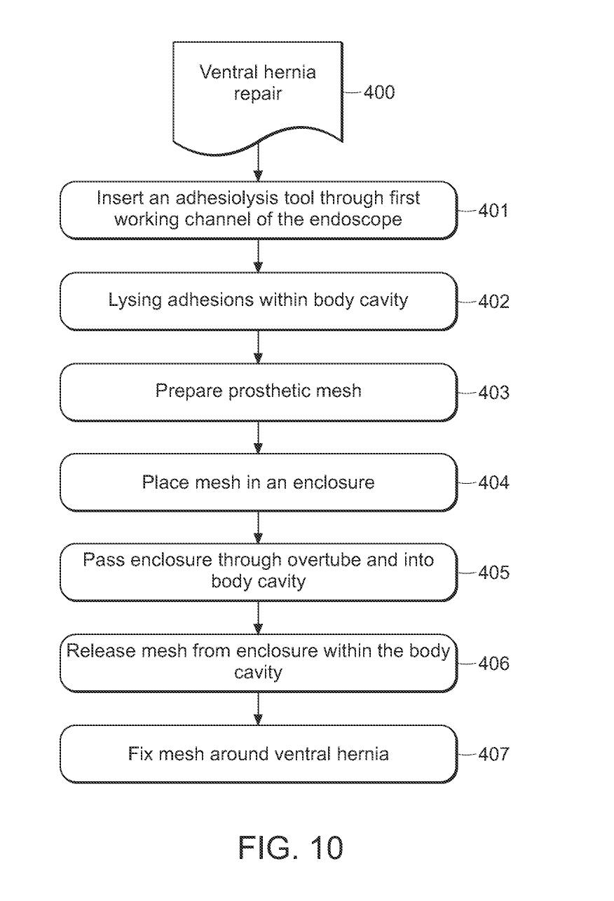

Focusing now on another exemplary embodiment, the specific surgical procedure 200 may be a ventral hernia repair 400, see FIGS. 8 and 10. Further to the surgical devices discussed above, additional devices which may be useful for a NOTES ventral hernia repair may include an adhesiolysis tool and/or an enclosure sized and configured to releasably contain a prosethetic mesh. One or more of these devices may also be a part of a surgical kit. Accordingly, in various embodiments, a surgical kit may include an overtube, an access device, and a tissue apposition device, as discussed above. Further, the surgical kit may also include an adhesiolysis tool and/or an enclosure configured to releasably contain a prosethetic mesh. Additional details regarding these instruments are provided below.

In at least one embodiment, the adhesiolysis tool may include endoscopic scissors. Such endoscopic scissors may be the same as or similar to the endoscopic scissors 71, see FIG. 14, described above. In another embodiment, the adhesiolysis tool may include endoscopic bipolar forceps. Such endoscopic bipolar forceps may be the same as or similar to the endoscopic bipolar forceps 73, see FIG. 16, also described above.

Further, in at least one embodiment, the enclosure may include a presterilized bag or pod for sterile delivery of a prosthetic surgical mesh to an operative site, within a patient's body cavity. As noted above, such a presterilized bag or pod may be configured to open within the body cavity, when actuated by a user, thereby enabling one to release the bag near a surgical site.

In various embodiments, the surgical kit for a ventral hernia repair procedure may include additional surgical instruments. For example, the surgical kit may further comprise a prosthetic mesh adaptable for repairing a ventral hernia. Additional, the surgical kit may comprise a suture passer. Prosthetic mesh for hernia repair and suture passers are known in the art and therefore additional details regarding their construction shall not be provided herein.

The above devices are just some of the surgical tools that may be part of a surgical kit used in a ventral hernia repair procedure. Moving now to the details of one such procedure, FIG. 10 illustrates a process flowchart showing an overview of some of the steps involved in a ventral hernia repair procedure 400, which may use some of the above described surgical tools. Details regarding any of the surgical tools mentioned below may be found in one or more of the embodiments discussed above. The steps leading up to those shown in FIG. 10 may include those of the access procedure 101 shown in FIG. 7, and discussed above. Thereafter, the steps of a ventral hernia repair procedure 400, according to at least one embodiment, may include the following. First, an adhesiolysis tool may be inserted through a first working channel of the first endoscope 401. In such embodiments, the adhesiolysis tool may also be inserted into a body cavity of the patient through the endoscope and/or overtube.

Second, the adhesiolysis tool may be used to lyse adhesions within the body cavity 402. In at least one embodiment, the adhesiolysis tool may include endoscopic scissors, as discussed above. In such an embodiment, the adhesions may be removed by cutting them with the scissors. Alternatively, the adhesiolysis tool may include flexible bipolar forceps, also as discussed above. In such an embodiment, the endoscopic bipolar forceps may be used to ablate the adhesions and/or to seal an artery and/or vein that need to be cut. Thus, in at least one embodiment, both endoscopic scissors and endoscopic bipolar forceps may be utilized to ablate and cut tissue. In any event, in at least one embodiment, an articulating grasper, inserted through a second endoscope working channel and into the body cavity, may be used to assist with manipulating and/or lysing the adhesions. Alternatively, in another embodiment, a laparoscopic trocar may be introduced through a tissue wall, such as the abdominal wall (see, e.g., abdominal wall 18 illustrated in FIG. 1), to introduce laparoscopic graspers to assist with the manipulation.

Third, a prosthetic mesh may be prepared for repairing a ventral hernia in the patient 403. As is known in the art, the mesh may be prepared using scissors and sutures to size and configure the mesh to repair the patient's ventral hernia. Sutures may be added to the perimeter of the mesh to later be used as anchors around the hernia defect.

Fourth, the prosthetic mesh may be placed in an enclosure 404. The enclosure, as discussed above, may be configured to releasably contain the prosthetic mesh and assist with sterile delivery thereof.

Fifth, the enclosure may be passed through the overtube and into the patient's body cavity 405. Passing the mesh within an enclosure is notably different than mesh delivery during a traditional laparoscopic ventral hernia repair. In a traditional procedure, the mesh is typically rolled upon itself and then fed through a laparoscopic trocar to the surgical site. However, in the present NOTES procedure, the mesh needs to pass through a complex path with other instruments nearby; further, the mesh and all of the other instruments are going through a restricted orifice. Therefore, the mesh could become contaminated if not properly enclosed during delivery.

Sixth, the prosthetic mesh may be released from the enclosure within the body cavity 406. Seventh, the mesh may be fixed around at least a portion of the ventral hernia 407. The mesh may be fixed using a suture passer and a stapling and/or tacking device as is known in the art. An exemplary suture passer is provided in U.S. patent application Ser. No. 08/074,321 to Failla et al., entitled “PERCUTANEOUS SUTURE EXTERNALIZER,” the disclosure of which is hereby incorporated by reference in its entirety. In at least one embodiment, the suture passer may be passed through the patient's body wall and then used to pull at least one suture attached to the prosthetic mesh with the suture passer. Thereafter the suture may be attached to the body wall to fix the mesh around at least a portion of the ventral hernia. Pulling and attaching the sutures may be repeated as necessary until all of the sutures are anchored to the body wall, thereby securing the mesh around the hernia defect.

After completing the ventral hernia repair procedure 400, the closure procedure 102 outlined in FIG. 7, and discussed above, may be executed to seal the incision and remove the surgical tools from the patient. Also, as mentioned above, the ordering of the above steps may be rearranged or two or more steps may be carried out contemporaneously as desired unless illogical or the order is explicitly required.

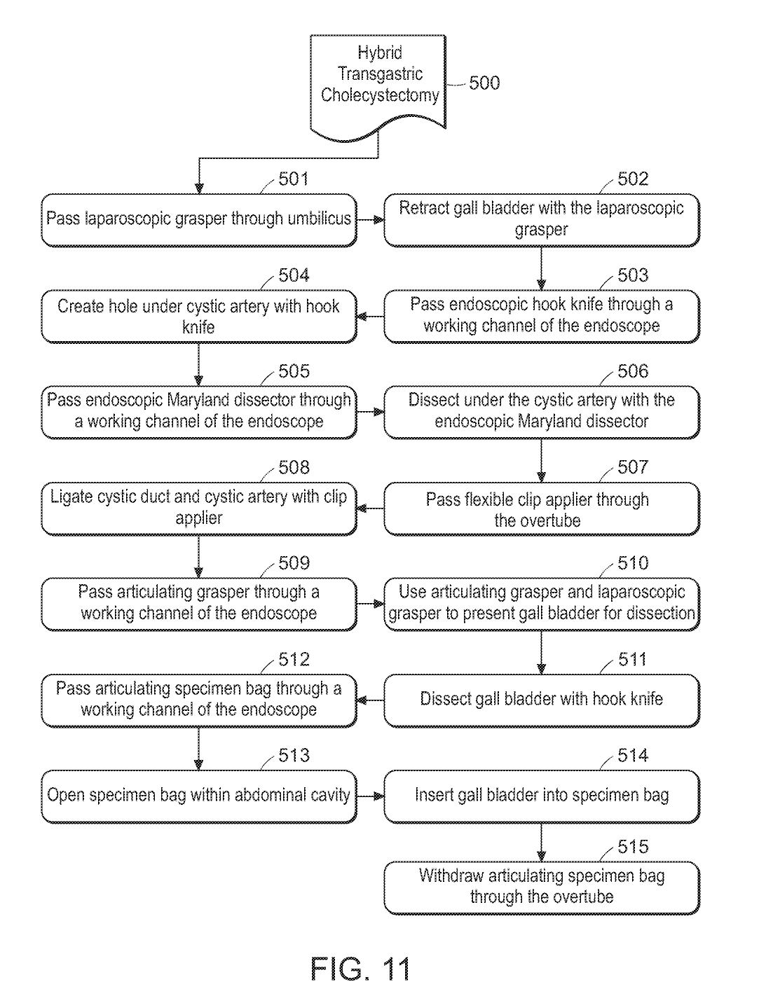

Focusing now on another exemplary embodiment, the specific surgical procedure 200 may be a hybrid transgastric cholecystectomy 500, see FIGS. 8 and 11. As used herein, a procedure termed as a “hybrid” procedure may include entry to a surgical site and/or body cavity through both a natural orifice and one or more traditional laparoscopic trocars inserted through a tissue wall, such as the abdominal wall (see, e.g., abdominal wall 18 illustrated in FIG. 1). Such hybrid procedures may reduce the number of laparoscopic trocars necessary to perform a procedure and thus may be advantageous to traditional laparoscopic procedures which require multiple trocars to pierce the abdominal wall, for example.

Further to the surgical devices discussed above, additional devices which may be useful for a hybrid transgastric cholecystectomy 500 may include an endoscopic hook knife, an endoscopic Maryland dissector, a flexible clip applier, an articulating grasper, and/or an articulating specimen bag. One or more of these devices may also be a part of a surgical kit. Accordingly, in various embodiments, a surgical kit may include an overtube, an access device, and a tissue apposition device, as discussed above. Further, the surgical kit may also include an endoscopic hook knife, an endoscopic Maryland dissector, a flexible clip applier, an articulating grasper, and/or an articulating specimen bag. Additional details regarding these instruments are provided below.

In at least one embodiment, the endoscopic hook knife may be the same or similar to articulating hook knife 72, see FIG. 15, described above. Alternatively, the endoscopic hook knife may be a non-articulating hook knife. In any event, the endoscopic hook knife may be sized and configured to fit through a working channel of an endoscope.

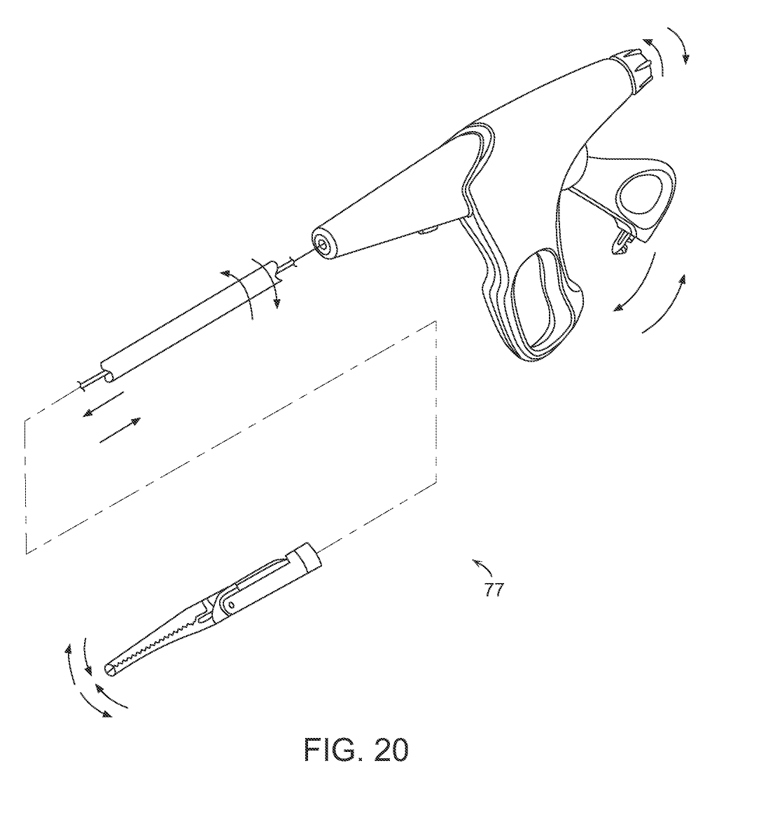

Also, in at least one embodiment, the endoscopic Maryland dissector may be endoscopic Maryland dissector 77, see FIG. 20. Endoscopic Maryland dissector 77 may be sized and configured to fit through the working channel of an endoscope. More details regarding endoscopic Maryland dissector 77 and other embodiments can be found in U.S. patent application Ser. No. 12/203,330, filed Sep. 3, 2008, entitled SURGICAL GRASPING DEVICE” to Matthew D. Holcomb et al., noted above. In such embodiments, the endoscopic Maryland dissector 77 may comprise a clevis defining a longitudinal axis, a jaw, a slider slidably engaged to the clevis, a driveline coupled to the slider, a handle portion to receive a proximal end of the driveline, and a trigger operatively coupled to the driveline. In more detail, the jaw may comprise a first member and a second member, and the first member may define a first slot. Further, the slider may comprise a pin and the pin may be receivably engaged in the first slot. The jaw may also be selectively moveable between a first position and a second position through longitudinal movement of the driveline. Additionally, the trigger may be pivotally moveable in a first rotational direction to move the driveline in the first direction to open the jaw, and the trigger may be pivotally moveable in a second rotational direction to move the driveline in the second direction to close the jaw;

In at least one embodiment, the flexible clip applier may be the same or similar as flexible clip applier 74, see FIGS. 17A-17B, described above. Also, in at least one embodiment, the articulating grasper may be the same or similar as articulating grasper 70, see FIGS. 13A-13B, described above. Further, in at least one embodiment, the articulating specimen bag may be the same or similar as articulating specimen bag 76, see FIG. 19, described above.

In various embodiments, the surgical kit for a hybrid transgastric cholecystectomy procedure may include additional surgical instruments. For example, the surgical kit may further comprise an articulating hook knife. The articulating hook knife may be the same or similar as articulating hook knife 72, see FIG. 15, described above. Also, in at least one embodiment, the surgical kit may further comprise endoscopic bipolar forceps. The endoscopic bipolar forceps may be the same or similar as endoscopic bipolar forceps 73, see FIG. 16, described above.

The above devices are just some of the surgical tools that may be part of a surgical kit used in a hybrid transgastric cholecystectomy. Moving now to the details of one such procedure, FIG. 11 illustrates a process flowchart showing an overview of some of the steps involved in a hybrid transgastric cholecystectomy 500, which may use some of the above described surgical tools. Details regarding any of the surgical tools mentioned below may be found in one or more of the embodiments discussed above. The steps leading up to those shown in FIG. 11 may include those of the access procedure 101 shown in FIG. 7, and discussed above. Thereafter, the steps of a hybrid transgastric cholecystectomy procedure 500, according to at least one embodiment, may include the following. First, as for any of the procedures described herein, it may be desirable to insufflate the patient's abdominal cavity using a Veress needle or other techniques as known in the art to provide increased working space within the body cavity. Second, a laparoscopic grasper may be passed through the patient's umbilicus 501. In at least one embodiment, a laparoscopic trocar, such as a traditional 5 mm laparoscopic trocar may be placed into the umbilicus using known laparoscopic techniques. Thereafter, the laparoscopic grasper, which may be a standard grasper known in the art, or another laparoscopic tool may be passed to the abdominal cavity through the trocar and, hence, through the umbilicus. Further, in at least one embodiment, a standard laparoscope may be inserted into the abdominal cavity before passing the laparoscopic grasper into the same such that various steps of the access procedure 101 shown in FIG. 7, and described above, may be better visualized. In such embodiments, one laparoscopic instrument, such as the laparoscopic grasper or the laparoscope, may be removed before inserting another laparoscopic instrument through the laparoscopic trocar.

Third, the steerable overtube may be articulated to allow the endoscope, discussed above, to visualize the patient's gall bladder. While the endoscope itself may have an articulatable portion, additional articulation may be provided by articulating the overtube also. For instance, see FIG. 5B, discussed above, which shows the overtube 40′ articulating in one direction, while the endoscope articulates in a second direction. Accordingly, the position and view provided by the endoscope may be enhanced with a steerable overtube such as overtube 40′ and/or overtube 40″, see FIG. 6A, for example.

Fourth, the gall bladder may be retracted with the laparoscopic grasper 502. Fifth, an endoscopic hook knife may be passed through a working channel of the endoscope 503. In at least one embodiment, the endoscopic hook knife may comprise a non-articulating hook knife. Alternatively, in another embodiment, the endoscopic hook knife may comprise an articulating hook knife. In any event, the hook knife may be configured to cut tissue. Sixth, a defect may be created in the tissue surrounding the cystic duct and artery with the hook knife 504.

Seventh, an endoscopic Maryland dissector may be passed through a working channel of the first endoscope 505. In at least one embodiment, the working channel may be the same as that used for the hook knife, discussed above. In such embodiments, the hook knife may be removed from the endoscope before introducing the Maryland dissector therethrough. Alternatively, in another embodiment, the endoscope may include multiple working channels such that the hook knife may be left in the endoscope while also passing the Maryland dissector therethrough. Eighth, the cystic artery and/or bundle may be dissected and isolated with the endoscopic Maryland dissector 506. In other words, surrounding tissue may be dissected away from the artery to “skeletonize” the artery to allow for clipping or sealing with endoscopic bipolar forceps, for example, and eventual cutting between the clips/seal lines, as discussed below.

Ninth, a flexible clip applier may be passed through the overtube 507. In at least one embodiment, the endoscope may be removed first and then the clip applier advanced through the overtube such that clips may be applied to tissue within the body cavity. To facilitate clip application, the flexible clip applier may further include a camera at its distal end such that a user may see the tissue to be ligated and to properly locate the clips within the body cavity. Tenth, the patient's cystic duct may be ligated with the flexible clip applier 508. Eleventh, the patient's cystic artery may likewise be ligated with the flexible clip applier 508. Alternatively, the cystic artery may be ligated with endoscopic bipolar forceps after replacing the clip applier with the endoscope such that the bipolar forceps may be guided to the surgical site through a working channel of the endoscope. In any event, the clip applier, if still present, may be removed and the endoscope passed back through the overtube before continuing.

Twelfth, an articulating grasper may be passed through a working channel of the endoscope 509. In at least one embodiment, the endoscopic bipolar forceps, if utilized, may be removed from the endoscope prior to inserting the articulating grasper therethrough. Thirteenth, the articulating grasper and the laparoscopic grasper may be used, in conjunction, to present the gall bladder for dissection from the liver bed 510.

Fourteenth, the gall bladder may be dissected with the endoscopic hook knife 511. In such embodiments, the endoscopic hook knife if inserted through a working channel of the endoscope to reach the gall bladder therethrough. As noted above, the endoscopic hook knife may be a non-articulating hook knife. Alternatively, the endoscopic hook knife may be an articulating hook knife. In any event, endoscopic graspers, such as standard 2.8 mm graspers, may be inserted through another working channel of the endoscope to further assist with dissecting the gall bladder from the liver bed.

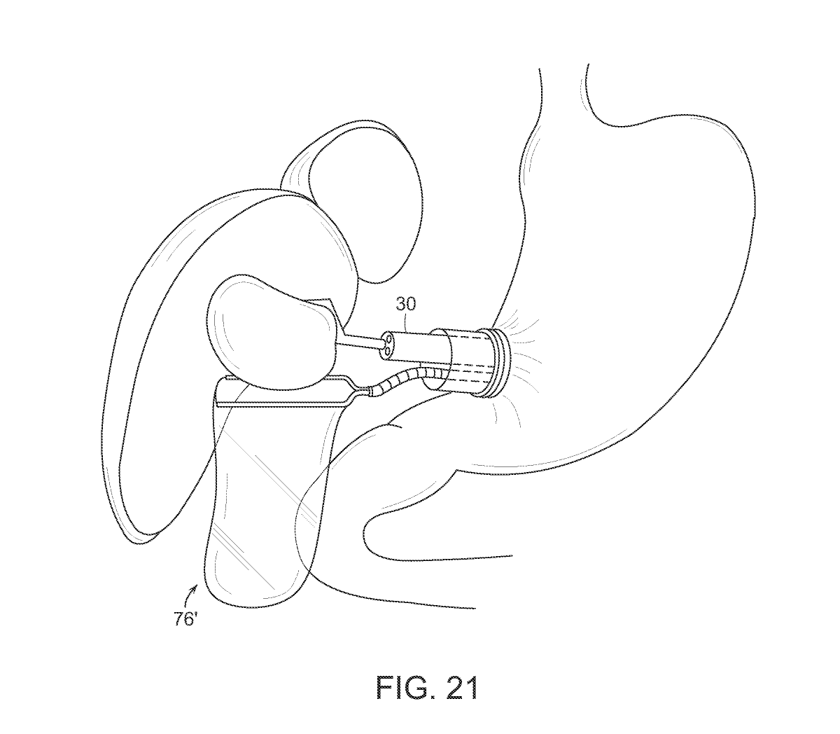

Fifteenth, an articulating specimen bag may be passed through a working channel of the endoscope 512. Sixteenth, the articulating specimen bag may be opened within the patient's abdominal or peritoneal cavity 513. Seventeenth, the gall bladder may be inserted into the articulating specimen bag 514. In at least one embodiment, an endoscopic grasper, such as an articulating grasper, may be inserted through another working channel of the endoscope to assist with directing, manipulating, and/or otherwise inserting the gall bladder into the specimen beg. Alternatively, in at least one embodiment, and referring to FIG. 21, the articulating specimen bag 76′ may be rigidizable and inserted into the body cavity next to the endoscope such that it may receive the gall bladder after dissection. Additional details regarding such a rigidizable specimen bag and other embodiments may be found in U.S. patent application Ser. No. 12/234,425, filed Sep. 19, 2008, entitled “RIGIDIZABLE SURGICAL INSTRUMENT” to Andrew M. Zwolinski et al., noted above. Eighteenth, the articulating specimen bag, which now contains the gall bladder, may be withdrawn through the steerable overtube 515.

After completing the hybrid transgastric cholecystectomy procedure 500, the closure procedure 102 outlined in FIG. 7, and discussed above, may be executed to seal the incision(s) and remove the surgical tools from the patient. Also, as mentioned above, the ordering of the above steps may be rearranged or two or more steps may be carried out contemporaneously as desired unless illogical or the order is explicitly required.

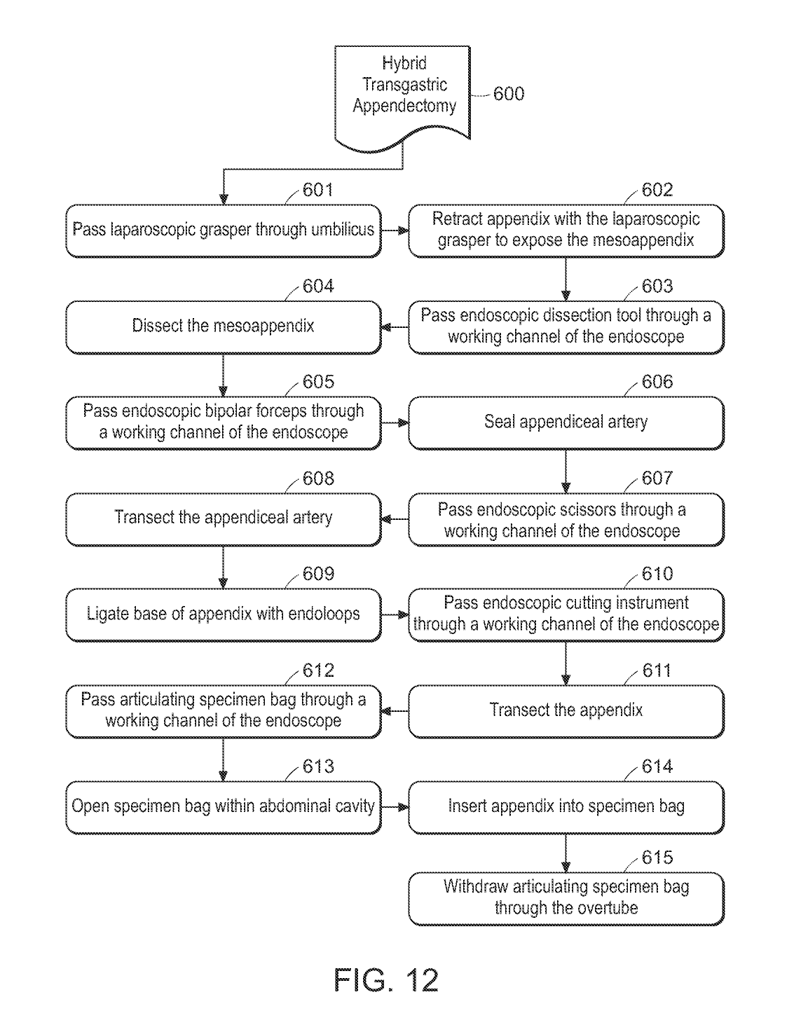

Focusing now on another exemplary embodiment, the specific surgical procedure 200 may be a hybrid transgastric appendectomy 600, see FIGS. 8 and 12. Further to the surgical devices discussed above, additional devices which may be useful for a hybrid transgastric appendectomy may include endoscopic bipolar forceps, endoscopic scissors, and/or an articulating specimen bag. One or more of these devices may also be a part of a surgical kit. Accordingly, in various embodiments, a surgical kit may include an overtube, an access device, and a tissue apposition device, as discussed above. Further, the surgical kit may also include endoscopic bipolar forceps, endoscopic scissors, and/or an articulating specimen bag. Additional details regarding these instruments are provided below.

In at least one embodiment, the endoscopic bipolar forceps may be the same or similar as endoscopic bipolar forceps 73, see FIG. 16, described above. Further, in at least one embodiment, the articulating specimen bag may be the same or similar as articulating specimen bag 76, see FIG. 19, described above. Also, in at least one embodiment, the endoscopic scissors may be the same as or similar to the endoscopic scissors 71, see FIG. 14, described above.

In various embodiments, the surgical kit for a hybrid transgastric appendectomy procedure may include additional surgical instruments. For example, the surgical kit may further comprise one or more endoscopic dissection tools. The endoscopic dissection tool may be configured to manipulate and/or cut tissue and may be sized and configured to fit through a working channel of an endoscope. In at least one such embodiment, the endoscopic dissection tool may include an endoscopic Maryland dissector. The endoscopic Maryland dissector may be the same or similar as endoscopic Maryland dissector 77, see FIG. 20, described above. In another embodiment, the endoscopic dissection tool may include an articulating grasper. The articulating grasper may be the same or similar as articulating grasper 70, see FIGS. 13A-13B, described above. In yet another embodiment, the endoscopic dissection tool may include an articulating hook knife. The articulating hook knife may be the same or similar as articulating hook knife 72, see FIG. 15, described above.

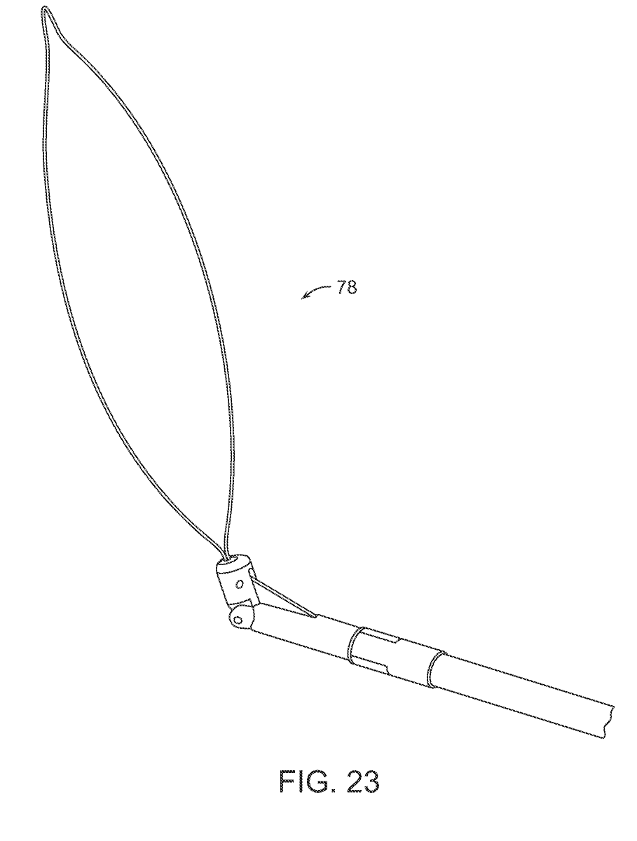

In at least one embodiment, the surgical kit for a hybrid transgastric appendectomy procedure may include an articulating snare loop, such as articulating snare loop 78 seen in FIG. 23. Articulating snare loop 78 may be sized and configured to fit through working channel of an endoscope. More details regarding articulating snare loop 78 and other embodiments can be found in U.S. patent application Ser. No. 11/610,803, filed Dec. 14, 2006, entitled “MANUALLY ARTICULATING DEVICES” to Rudolph H. Nobis et al., the disclosure of which is incorporated herein by reference in its entirety. In such embodiments, the articulating snare loop 78 may comprise an elongate shaft having proximal and distal ends, a three-bar linkage having proximal and distal ends, with the proximal end being coupled to the distal end of the elongate shaft, a snare loop coupled to the distal end of the three-bar linkage, and an articulation actuator extending through the elongate shaft and effective to laterally articulate the three-bar linkage relative to a longitudinal axis of the elongate shaft to angularly orient the end effector relative to the elongate shaft.

The above devices are just some of the surgical tools that may be part of a surgical kit used in a hybrid transgastric appendectomy. Moving now to the details of one such procedure, FIG. 12 illustrates a process flowchart showing an overview of some of the steps involved in a hybrid transgastric appendectomy 600, which may use some of the above described surgical tools. Details regarding any of the surgical tools mentioned below may be found in one or more of the embodiments discussed above. The steps leading up to those shown in FIG. 12 may include those of the access procedure 101 shown in FIG. 7, and discussed above. Thereafter, the steps of a hybrid transgastric appendectomy procedure 600, according to at least one embodiment, may include the following. First, similar to that described above, the patient's abdominal cavity may be insufflated using a Veress needle or other techniques as known in the art to provide increased working space within the body cavity. Second, a laparoscopic grasper may be passed through the patient's umbilicus 601. Similar to that described above, in at least one embodiment, a laparoscopic trocar, such as a traditional 5 mm laparoscopic trocar may be placed into the umbilicus using known laparoscopic techniques. Thereafter, the laparoscopic grasper, which may be a standard grasper known in the art, or another laparoscopic tool may be passed to the abdominal cavity through the trocar and, hence, through the umbilicus. Further, in at least one embodiment, a standard laparoscope may be inserted into the abdominal cavity before passing the laparoscopic grasper into the same such that various steps of the access procedure 101 shown in FIG. 7, and described above, may be better visualized. In such embodiments, one laparoscopic instrument, such as the laparoscopic grasper or the laparoscope, may be removed before inserting another laparoscopic instrument through the laparoscopic trocar.

Third, similar to that described above for visualizing a patient's gall bladder, the steerable overtube may be articulated to allow the endoscope to visualize the patient's appendix.

Fourth, the appendix may be retracted with the laparoscopic grasper to expose the mesoappendix 602. In at least one embodiment, the laparoscopic graspers may be used to locate and retract the appendix, by running along the bowel, thereby exposing the mesoappendix. As used herein, the phrase “running along the bowel” is a shorthand term used to describe the process of grasping the bowel with one grasper/forceps, pulling and/or moving it with the forceps under visualization in a direction that brings more bowel into the visual field, grasping the bowel at a position closer to the target location with a second pair of forceps, releasing the first forceps, pulling/moving the bowel with the second forceps, then re-grasping at a location closer to the target with the first forceps and repeating until reaching the target, in this case, the appendix.

Fifth, an endoscopic dissection tool may be passed through a working channel of the endoscope 603. In various embodiments, the endoscopic dissection tool may be one or more of the following: an endoscopic Maryland dissector, an articulating grasper, an endoscopic hook knife, endoscopic bipolar forceps, endoscopic scissors, and/or any other device sized and configured to fit through a working channel of an endoscope to manipulate and/or cut tissue within a body cavity. Sixth, the mesoappendix may be dissected 604. In at least one embodiment, one or more endoscopic dissection tools, as listed above, may be used to dissect the mesoappendix.

Seventh, endoscopic bipolar forceps may be passed through a working channel of the endoscope 605. Eighth, the appendiceal artery may be sealed 606. In at least one embodiment, the endoscopic bipolar forceps may be used to seal the appendiceal artery.

Ninth, endoscopic scissors may be passed through a working channel of the endoscope 607. Tenth, the appendiceal artery may be transected 608. In at least one embodiment, the endoscopic scissors may be used to transect or cut the appendiceal artery.

Eleventh, the base of the appendix may be ligated with endoloops 609. Alternatively, other known ligation techniques and/or devices may be used in place of the endoloops.

Twelfth, an endoscopic cutting instrument may be passed through a working channel of the endoscope 610. In various embodiments, the endoscopic cutting instrument may be one or more of the following: an articulating snare loop, an articulating hook knife, endoscopic scissors, and/or any other device sized and configured to fit through a working channel of an endoscope to cut tissue within a body cavity. Thirteenth, the appendix may be transected 611. In at least one embodiment, one or more endoscopic cutting instruments, as listed above, may be used to transect or cut the appendix.

Fourteenth, an articulating specimen bag may be passed through a working channel of the endoscope 612. Fifteenth, the articulating specimen bag may be opened within the patient's abdominal cavity 613. Sixteenth, the appendix may be inserted into the articulating specimen bag 614. In at least one embodiment, an endoscopic grasper, such as an articulating grasper, may be inserted through another working channel of the endoscope to assist with directing, manipulating, and/or otherwise inserting the gall bladder into the specimen beg. Seventeenth, the articulating specimen bag containing the appendix may be withdrawn through the steerable overtube 615.

After completing the hybrid transgastric appendectomy procedure 600, the closure procedure 102 outlined in FIG. 7, and discussed above, may be executed to seal the incision(s) and remove the surgical tools from the patient. Also, as mentioned above, the ordering of the above steps may be rearranged or two or more steps may be carried out contemporaneously as desired unless illogical or the order is explicitly required.

It is understood that where applicable above, if an endoscopic instrument or another tool is needed to pass through a working channel of the endoscope, and that working channel is occupied by another instrument, then the latter instrument may be removed first from the working channel before inserting the former instrument. However, in various embodiments, an endoscope may have multiple working channels, and in such embodiments, one endoscopic instrument may remain in a first working channel while a second endoscopic instrument may be inserted through a second, and so forth until all of the working channels are occupied.

While the embodiments have been described, it should be apparent, however, that various modifications, alterations and adaptations to the embodiments may occur to persons skilled in the art with the attainment of some or all of the advantages of the various embodiments. For example, according to various embodiments, a single component or step may be replaced by multiple components or steps, and multiple components or steps may be replaced by a single component or step, to perform a given function or functions or accomplish a given objective. This application is therefore intended to cover all such modifications, alterations and adaptations without departing from the scope and spirit of the appended claims.

The devices disclosed herein can be designed to be disposed of after a single use, or they can be designed to be used multiple times. In either case, however, the devices can be reconditioned for reuse after at least one use. Reconditioning can include a combination of the steps of disassembly of the device, followed by cleaning or replacement of particular pieces, and subsequent reassembly. In particular, the devices can be disassembled, and any number of particular pieces or parts of the device can be selectively replaced or removed in any combination. Upon cleaning and/or replacement of particular parts, the devices can be reassembled for subsequent use either at a reconditioning facility, or by a surgical team immediately prior to a surgical procedure. Those of ordinary skill in the art will appreciate that the reconditioning of a device can utilize a variety of different techniques for disassembly, cleaning/replacement, and reassembly. Use of such techniques, and the resulting reconditioned device, are all within the scope of the present application.

The devices described herein may be processed before surgery. First a new or used instrument is obtained and, if necessary, cleaned. The instrument can then be sterilized. In one sterilization technique, the instrument is placed in a closed and sealed container, such as a plastic or TYVEK® bag. The container and instrument are then placed in a field of radiation that can penetrate the container, such as gamma radiation, x-rays, or higher energy electrons. The radiation kills bacteria on the instrument and in the container. The sterilized instrument can then be stored in the sterile container. The sealed container keeps the instrument sterile until it is opened in the medical facility.

Any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

What is claimed is:

1. A surgical kit, comprising:
a guide system for accommodating endoscopic tools, the guide system comprising:
a hollow overtube having a proximal end and a distal end, the distal end being substantially steerable; and
an inner sheath having a proximal end and a distal end and being sized relative to the hollow overtube to permit the inner sheath to be selectively rotated and axially moved within the hollow overtube such that the distal end of the inner sheath may selectively protrude beyond the distal end of the hollow overtube and wherein the inner sheath has at least one working channel formed therein, wherein a distal end of the at least one working channel is substantially steerable;
a translumenal access device comprising:
a catheter comprising a proximal end, a distal end, at least one first lumen, and at least one second lumen, the at least one first lumen configured to slidably receive a guide wire from the proximal end to the distal end of the catheter;
an inflatable member mounted near the distal end of the catheter and in fluid communication with the second lumen;
a hollow needle mounted on the distal end of the catheter and mounted distal to the inflatable member;
a stylet comprising a third lumen, the stylet configured to be slidably disposed within the hollow needle, and the stylet comprising at least one extended position and at least one retracted position; and
a guide wire slidably moveable between an extended position and a retracted position, wherein in the extended position, the guide wire is extended distally from the stylet and in the retracted position, the guide wire is retracted proximally from the stylet, and wherein the guide wire is configured to be received in at least a part of the first lumen and at least a part of the third lumen; and
an expandable suture anchor comprising:
a body defining a longitudinal axis and a bifurcated portion, the bifurcated portion defining at least two legs, each of the legs including a first portion that is generally parallel with the longitudinal axis and a second portion that is splayed at an angle relative to the longitudinal axis; and
a suture connected to the body, wherein the suture extends at least partially through the bifurcated portion.

2. The surgical kit of claim 1, further comprising:
an articulating grasper comprising:
an elongate shaft having proximal and distal ends;
a three-liar linkage having proximal and distal ends, the proximal end being coupled to the distal end of the elongate shaft;
a grasper coupled to the distal end of the three-bar linkage; and
an articulation actuator extending through the elongate shaft and effective to laterally articulate the three-bar linkage relative to a longitudinal axis of the elongate shaft to angularly orient the grasper relative to the elongate shaft;
an endoscopic cutting instrument; and
an endocutter configured to cut and seal tissue.

3. The surgical kit of claim 2, wherein the endoscopic cutting instrument comprises endoscopic scissors, the endoscopic scissors comprising:
a clevis comprising a pair of arms;
a first blade member comprising a first distally positioned blade end and a first proximally positioned cam, wherein the first cam defines a first cam slot;
a second blade member comprising a second distally positioned blade and a second proximally positioned cam defining a second cam slot;
a fastener positioned to pivotably couple the first blade member and the second blade member to the clevis about a pivot point, wherein the fastener is held in tension by the clevis;
a reciprocating shuttle comprising at least one pin positioned within the first cam slot and the second cam slot such that distally-directed motion of the shuttle causes the first and second blade members to open and proximally-directed motion of the shuttle causes the first and second blade members to close;
a handle comprising an actuator selectively positionable in a first position and a second position;
a flexible shaft; and
a translating member coupled to the shuttle, extending through the flexible shaft and coupled to the actuator such that placing the actuator in the first position causes the shuttle to translate distally and placing the actuator in the second position causes the shuttle to translate proximally.

4. The surgical kit of claim 2, wherein the endoscopic cutting instrument comprises an articulating hook knife, the articulating hook knife comprising:
an elongate shaft comprising proximal and distal ends;
an articulation joint comprising proximal and distal ends, the proximal end coupled to the distal end of the elongate shaft;
an actuation wire extending through the elongate shaft and the articulation joint; and
an end effector, comprising:
a distal tip coupled to the distal end of the articulation joint, the distal tip receiving therethrough a distal end of the actuation wire;
a hook knife disposed adjacent the distal tip and comprising proximal and distal ends, the proximal end of the hook knife attached to the distal end of the actuation wire;
wherein the actuation wire is translatable along a longitudinal axis of the elongate shaft to extend and retract the distal end of the hook knife relative to the distal tip; and
wherein the articulation joint is articulatable relative to the longitudinal axis of the elongate shaft to allow the end effector to be angularly oriented relative to the elongate shaft.

5. The surgical kit of claim 2, further comprising endoscopic bipolar forceps, the endoscopic bipolar forceps comprising:
an elongated flexible member having a proximal end and a distal end, the flexible member comprising at least one lumen;
a clevis coupled to the elongated flexible member;
first and second jaw members pivotally coupled to the clevis forming a clamp jaw, the first and second jaw members comprising respective first and second electrodes to couple to an electrical waveform generator; and
an elongated actuator member slidably received within the at least one lumen, the elongated actuator member coupled to the clevis, wherein longitudinal motion of the elongated actuator element in a first longitudinal direction opens the first and second jaw members and longitudinal motion in a second opposite direction closes the first and second jaw members;
wherein the first and second electrodes are adapted to couple to an electrical waveform generator and to receive an electrical waveform sufficient to electrically ablate tissue located between the first and second jaw members.

6. The surgical kit of claim 2, further comprising a flexible clip applier, the flexible clip applier comprising:
an elongate clip magazine having an axial clip passage therein for receiving a plurality of tissue apposition clips therein;
at least one grasper lumen in the elongate clip magazine apart from the axial clip passage and configured to movably accommodate a corresponding grasper device therethrough to manipulate tissue relative to a distal end of the elongate clip magazine; and
an advancement member for applying an advancement motion to the tissue apposition clips in the axial passage to cause the tissue apposition clips to move out of the axial clip passage in seriatim.

7. The surgical kit of claim 2, further comprising an articulating needle knife, the articulating needle knife comprising:
an elongate shaft having proximal and distal ends;
a three-bar linkage having proximal and distal ends, the proximal end being coupled to the distal end of the elongate shaft;
a needle knife coupled to the distal end of the three-bar linkage; and
an articulation actuator extending through the elongate shaft and effective to laterally articulate the three-bar linkage relative to a longitudinal axis of the elongate shaft to angularly orient the needle knife relative to the elongate shaft.

8. The surgical kit of claim 1, further comprising:
an adhesiolysis tool; and
an enclosure sized and configured to deliver a prosethetic mesh to a body cavity.

9. The surgical kit of claim 8, wherein the adhesiolysis tool includes endoscopic scissors comprising:
a clevis comprising a pair of arms;
a first blade member comprising a first distally positioned blade end and a first proximally positioned cam, wherein the first cam defines a first cam slot;
a second blade member comprising a second distally positioned blade and a second proximally positioned cam defining a second cam slot;
a fastener positioned to pivotably couple the first blade member and the second blade member to the clevis about a pivot point, wherein the fastener is held in tension by the clevis;
a reciprocating shuttle comprising at least one pin positioned within the first cam slot and the second cam slot such that distally-directed motion of the shuttle causes the first and second blade members to open and proximally-directed motion of the shuttle causes the first and second blade members to close;
a handle comprising an actuator selectively positionable in a first position and a second position;
a flexible shaft; and
a translating member coupled to the shuttle, extending through the flexible shaft and coupled to the actuator such that placing the actuator in the first position causes the shuttle to translate distally and placing the actuator in the second position causes the shuttle to translate proximally.

10. The surgical kit of claim 8, wherein the adhesiolysis tool includes endoscopic bipolar forceps comprising:
an elongated flexible member having a proximal end and a distal end, the flexible member comprising at least one lumen;
a clevis coupled to the elongated flexible member;
first and second jaw members pivotally coupled to the clevis forming a clamp jaw, the first and second jaw members comprising respective first and second electrodes to couple to an electrical waveform generator; and
an elongated actuator member slidably received within the at least one lumen, the elongated actuator member coupled to the clevis, wherein longitudinal motion of the elongated actuator element in a first longitudinal direction opens the first and second jaw members and longitudinal motion in a second opposite direction closes the first and second jaw members;
wherein the first and second electrodes are adapted to couple to an electrical waveform generator and to receive an electrical waveform sufficient to electrically ablate tissue located between the first and second jaw members.

11. The surgical kit of claim 1, further comprising
an endoscopic hook knife;
an endoscopic Maryland dissector comprising:
a clevis defining a longitudinal axis;
a jaw comprising a first member and a second member, the first member defining a first slot;
a slider slidably engaged to the clevis, the slider comprising a pin;
a driveline coupled to the slider, wherein the pin is receivably engaged in the first slot and the jaw is selectively moveable between a first position and a second position through longitudinal movement of the driveline;
a handle portion to receive a proximal end of the driveline;
a trigger operatively coupled to the driveline;
wherein the trigger is pivotally moveable in a first rotational direction to move the driveline in the first direction to open the jaw; and
wherein the trigger is pivotally moveable in a second rotational direction to move the driveline in the second direction to close the jaw;
a flexible clip applier comprising:
an elongate clip magazine having an axial clip passage therein for receiving a plurality of tissue apposition clips therein;
at least one grasper lumen in the elongate clip magazine apart from the axial clip passage and configured to movably accommodate a corresponding grasper device therethrough to manipulate tissue relative to a distal end of the elongate clip magazine; and
an advancement member for applying an advancement motion to the tissue apposition clips in the axial passage to cause the tissue apposition clips to move out of the axial clip passage in seriatim;
an articulating grasper comprising:
an elongate shaft having proximal and distal ends;
a three-bar linkage having proximal and distal ends, the proximal end being coupled to the distal end of the elongate shaft;
a grasper coupled to the distal end of the three-bar linkage; and
an articulation actuator extending through the elongate shaft and effective to laterally articulate the three-bar linkage relative to a longitudinal axis of the elongate shaft to angularly orient the grasper relative to the elongate shaft; and
an articulating specimen bag comprising:
a hybrid shaft having a proximal end and a distal end, wherein the hybrid shaft extends from a proximal handle to the distal end of the articulating specimen bag, wherein the distal end is flexible, and wherein the proximal end is rigid;
at least one collapsible arm located at the distal end of the hybrid shaft;
a bag having an open end and a closed end, wherein the bag is configured to be retained upon the at least one collapsible arm;
a knot pusher located at the distal end of the hybrid shaft;
an articulating joint, wherein the articulating joint connects the at least one collapsible arm to the hybrid shaft; and
an outer sheath extending from a distal handle to the distal end of the articulating specimen bag.

12. The surgical kit of claim 11, wherein the endoscopic hook knife comprises an articulating hook knife, the articulating hook knife comprising:
an elongate shaft comprising proximal and distal ends;
an articulation joint comprising proximal and distal ends, the proximal end coupled to the distal end of the elongate shaft;
an actuation wire extending through the elongate shaft and the articulation joint; and
an end effector, comprising:
a distal tip coupled to the distal end of the articulation joint, the distal tip receiving therethrough a distal end of the actuation wire; and
a hook knife disposed adjacent the distal tip and comprising proximal and distal ends, the proximal end of the hook knife attached to the distal end of the actuation wire;
wherein the actuation wire is translatable along a longitudinal axis of the elongate shaft to extend and retract the distal end of the hook knife relative to the distal tip; and
wherein the articulation joint is articulatable relative to the longitudinal axis of the elongate shaft to allow the end effector to be angularly oriented relative to the elongate shaft.

13. The surgical kit of claim 11, further comprising endoscopic bipolar forceps, the endoscopic bipolar forceps comprising:
an elongated flexible member having a proximal end and a distal end, the flexible member comprising at least one lumen;
a clevis coupled to the elongated flexible member;
first and second jaw members pivotally coupled to the clevis forming a clamp jaw, the first and second jaw members comprising respective first and second electrodes to couple to an electrical waveform generator; and
an elongated actuator member slidably received within the at least one lumen, the elongated actuator member coupled to the clevis, wherein longitudinal motion of the elongated actuator element in a first longitudinal direction opens the first and second jaw members and longitudinal motion in a second opposite direction closes the first and second jaw members;
wherein the first and second electrodes are adapted to couple to an electrical waveform generator and to receive an electrical waveform sufficient to electrically ablate tissue located between the first and second jaw members.

14. The surgical kit of claim 1, further comprising:
endoscopic bipolar forceps comprising:
an elongated flexible member having a proximal end and a distal end, the flexible member comprising at least one lumen;
a clevis coupled to the elongated flexible member;
first and second jaw members pivotally coupled to the clevis forming a clamp jaw, the first and second jaw members comprising respective first and second electrodes to couple to an electrical waveform generator; and
an elongated actuator member slidably received within the at least one lumen, the elongated actuator member coupled to the clevis, wherein longitudinal motion of the elongated actuator element in a first longitudinal direction opens the first and second jaw members and longitudinal motion in a second opposite direction closes the first and second jaw members;
wherein the first and second electrodes are adapted to couple to an electrical waveform generator and to receive an electrical waveform sufficient to electrically ablate tissue located between the first and second jaw members;
endoscopic scissors comprising:
a clevis comprising a pair of arms;
a first blade member comprising a first distally positioned blade end and a first proximally positioned cam, wherein the first cam defines a first cam slot;
a second blade member comprising a second distally positioned blade and a second proximally positioned cam defining a second cam slot;
a fastener positioned to pivotably couple the first blade member and the second blade member to the clevis about a pivot point, wherein the fastener is held in tension by the clevis;
a reciprocating shuttle comprising at least one pin positioned within the first cam slot and the second cam slot such that distally-directed motion of the shuttle causes the first and second blade members to open and proximally-directed motion of the shuttle causes the first and second blade members to close;
a handle comprising an actuator selectively positionable in a first position and a second position;
a flexible shaft; and
a translating member coupled to the shuttle, extending through the flexible shaft and coupled to the actuator such that placing the actuator in the first position causes the shuttle to translate distally and placing the actuator in the second position causes the shuttle to translate proximally; and
an articulating specimen bag comprising:
a hybrid shaft having a proximal end and a distal end, wherein the hybrid shaft extends from a proximal handle to the distal end of the articulating specimen bag, wherein the distal end is flexible, and wherein the proximal end is rigid;
at least one collapsible arm located at the distal end of the hybrid shaft;
a bag having an open end and a closed end, wherein the bag is configured to be retained upon the at least one collapsible arm;
a knot pusher located at the distal end of the hybrid shaft;
an articulating joint, wherein the articulating joint connects the at least one collapsible arm to the hybrid shaft; and
an outer sheath extending from a distal handle to the distal end of the articulating specimen bag.

15. The surgical kit of claim 14, further comprising an articulating snare loop, the articulating snare loop comprising:
an elongate shaft having proximal and distal ends;
a three-bar linkage having proximal and distal ends, the proximal end being coupled to the distal end of the elongate shaft;
a snare loop coupled to the distal end of the three-bar linkage; and
an articulation actuator extending through the elongate shaft and effective to laterally articulate the three-bar linkage relative to a longitudinal axis of the elongate shaft to angularly orient the snare loop relative to the elongate shaft.

16. A surgical method, comprising:
obtaining a steerable overtube comprising a body defining a lumen therethrough, wherein the body includes a distal portion and a proximal portion;
placing an insertable portion of a first endoscope into the overtube's lumen, wherein the first endoscope includes at least one working channel;
inserting the overtube's distal portion and the endoscope's insertable portion into a patient's natural orifice;
positioning a portion of a translumenal access device through the working channel of the first endoscope, wherein the access device comprises a needle and an inflatable member mounted near the needle;
puncturing a tissue wall within the patient with the needle to create an incision;
locating the inflatable member within the incision;
inflating the inflatable member to dilate the incision;
passing the overtube's distal portion and the endoscope's insertable portion through the dilated incision;
performing a specific surgical procedure within the patient;
moving the overtube's distal portion and the endoscope's insertable portion out of the dilated incision;
sealing the incision; and
removing the steerable overtube and the first endoscope from the patient.

17. The surgical method of claim 16, wherein the natural orifice is the colon or the vagina, and wherein the specific surgical procedure comprises:
placing a second endoscope through the patient's mouth and into the patient's stomach;
passing a laparoscopic grasper through the patient's umbilicus;
inserting an articulating grasper through a first working channel of the first endoscope;
inserting an endoscopic cutting instrument through a second working channel of the first endoscope;
creating an opening in the patient's omentum with the endoscopic grasper and the endoscopic cutting instrument;
placing a laparoscope through the patient's umbilicus;
placing an endocutter configured to cut and seal tissue through the overtube's lumen;
resecting a portion of the patient's stomach with the endocutter to form a gastric remnant, wherein the second endoscope provides a guide for the endocutter;
mobilizing the gastric remnant from the patient's vasculature; and
removing the gastric remnant from the patient.

18. The surgical method of claim 16, wherein the specific surgical procedure comprises:
inserting an adhesiolysis tool through a first working channel of the first endoscope and into a body cavity of the patient;
lysing adhesions within the body cavity with the adhesiolysis tool;
preparing a prosthetic mesh for repairing a ventral hernia in the patient;
placing the prosthetic mesh in an enclosure;
passing the enclosure containing the prosthetic mesh through the overtube and into the body cavity;
releasing the prosthetic mesh from the enclosure within the body cavity; and
fixing the mesh around at least a portion of the ventral hernia.

19. The surgical method of claim 16, wherein the specific surgical procedure comprises:
insufflating the patient's abdominal cavity;
passing a laparoscopic grasper through the patient's umbilicus;
articulating the steerable overtube to allow the first endoscope to visualize the patient's gall bladder;
retracting the gall bladder with the laparoscopic grasper;
passing an endoscopic hook knife through a working channel of the first endoscope;
creating a hole in the patient's peritoneum under the cystic artery with the hook knife;
passing an endoscopic Maryland dissector through a working channel of the first endoscope;
dissecting under the cystic artery with the endoscopic Maryland dissector;
passing a flexible clip applier through the steerable overtube;
ligating the patient's cystic duct with the flexible clip applier;
ligating the patient's cystic artery with the flexible clip applier;
passing an articulating grasper through a working channel of the first endoscope;
using the articulating grasper and the laparoscopic grasper to present the gall bladder for dissection;
dissecting the gall bladder with the endoscopic hook knife;
passing an articulating specimen bag through a working channel of the first endoscope;
opening the articulating specimen bag within the patient's abdominal cavity;
inserting the gall bladder into the articulating specimen bag; and
withdrawing the articulating specimen bag containing the gall bladder through the steerable overtube.

20. The surgical method of claim 16, wherein the specific surgical procedure comprises:
insufflating the patient's abdominal cavity;
passing a laparoscopic grasper through the patient's umbilicus;
articulating the steerable overtube to allow the first endoscope to visualize the patient's appendix;
retracting the appendix with the laparoscopic grasper to expose the mesoappendix;
passing an endoscopic dissection tool through a working channel of the first endoscope;
dissecting the mesoappendix with the endoscopic dissection tool;
passing endoscopic bipolar forceps through a working channel of the first endoscope;
sealing the appendiceal artery with the endoscopic bipolar forceps;
passing endoscopic scissors through a working channel of the first endoscope;
transecting the appendicieal artery;
ligating the base of the appendix with endoloops;
passing an endoscopic cutting instrument through a working channel of the first endoscope;
transecting the appendix with the endoscopic cutting instrument;
passing an articulating specimen bag through a working channel of the first endoscope;
opening the articulating specimen bag within the patient's abdominal cavity;
inserting the appendix into the articulating specimen bag; and
withdrawing the articulating specimen bag containing the appendix through the steerable overtube.
